SEQUENCE LISTING (54 sequences)

SEQ ID NO: 1 - protein, 24 aa, Influenzavirus A,B (Human influenza virus)
MSLLTEVETPIRNEWGCRCNDSSD

SEQ ID NO: 2 - protein, 8 aa, Influenzavirus A,B (Avian influenza virus)
EVETPTRN

SEQ ID NO: 3 - protein, 8 aa, Influenzavirus A,B (Avian influenza virus)
EVETLTRN

SEQ ID NO: 4 - protein, 16 aa, Rhinovirus (Human rhinovirus)
NTEPVIKKRKGDIKSY

SEQ ID NO: 5 - protein, 24 aa, Influenzavirus A, B (Human influenza virus)
MSLLTEVETPTRNEWECRCSDSSD

SEQ ID NO: 6 - protein, 24 aa, Influenzavirus A,B (Avian influenza virus; Human influenza virus)
MSLLTEVETLTRNGWGCRCSDSSD

SEQ ID NO: 7 - protein, 23 aa, Influenzavirus A,B (Human influenza virus)
SLLTEVETPIRNEWGCRCNDSSD

SEQ ID NO: 8 - protein, 17 aa, Influenzavirus A,B (Human influenza virus)
SLLTEVETPIRNEWGCR

SEQ ID NO: 9 - protein, 6 aa, Influenzavirus B (Human influenza virus)
MLEPFQ

SEQ ID NO: 10 - protein, 20 aa, Influenzavirus B (Human influenza virus)
MNNATFNYTNVNPISHIRGS

SEQ ID NO: 11 - protein, 20 aa, Papillomavirus (Human papilloma virus 16)
QLYKTCKQAGTCPPDIIPKV

SEQ ID NO: 12 - protein, 26 aa, Artificial Sequence
  Synthetic
GHTSLLKEVETPIRNEWGSRSNDSSD

SEQ ID NO: 13 - protein, 20 aa, Artificial Sequence
  Synthetic
QPASLLTEVETPIRNEWGSR

SEQ ID NO: 14 - protein, 8 aa, Flavivirus (Hepatitis C virus)
STNPKPQR

SEQ ID NO: 15 - protein, 10 aa, Flavivirus (Hepatitis C virus)
YLLPRRGPRL

SEQ ID NO: 16 - protein, 9 aa, Flavivirus (Hepatitis C virus)
GPRLGVRAT

SEQ ID NO: 17 - protein, 20 aa, Flavivirus (Hepatitis C virus)
YPWPLYGNEGCGWAGWLLSP

SEQ ID NO: 18 - protein, 16 aa, Flavivirus (Hepatitis C virus)
GFADLMGYIPLVGAPL

SEQ ID NO: 19 - protein, 9 aa, Flavivirus (Hepatitis C virus)
DLMGYIPLV

SEQ ID NO: 20 - protein, 10 aa, Flavivirus (Hepatitis C virus)
LLALLSCLTV

SEQ ID NO: 21 - protein, 20 aa, Flavivirus (Hepatitis C virus)
REGNASRCWVAVTPTVATRD

SEQ ID NO: 22 - protein, 9 aa, Flavivirus (Hepatitis C virus)
STGLIHLHQ

SEQ ID NO: 23 - protein, 10 aa, Flavivirus (Hepatitis C virus)
LLADARVCSC

SEQ ID NO: 24 - protein, 11 aa, Flavivirus (Hepatitis C virus)
CWHYPPRPCGI

SEQ ID NO: 25 - protein, 10 aa, Flavivirus (Hepatitis C virus)
CVIGGVGNNT

SEQ ID NO: 26 - protein, 10 aa, Flavivirus (Hepatitis C virus)
RRLTDFAQGW

SEQ ID NO: 27 - protein, 8 aa, Flavivirus (Hepatitis C virus)
TINYTIFK

SEQ ID NO: 28 - protein, 27 aa, Flavivirus (Hepatitis C virus)
ETHVTGGNAGRTTAGLVGLLTPGAKQN

SEQ ID NO: 29 - protein, 27 aa, Flavivirus (Hepatitis C virus)
IQLINTNGSWHINSTALNCNESLNTGW

SEQ ID NO: 30 - protein, 20 aa, Flavivirus (Hepatitis C virus)
LFYQHKFNSSGCPERLASCR

SEQ ID NO: 31 - protein, 36 aa, Flavivirus (Hepatitis C virus)
PSPVVVGTTDRSGAPTYSWGANDTDVFVLNNTRPPL

SEQ ID NO: 32 - protein, 6 aa, Flavivirus (Hepatitis C virus)
IQLINT

SEQ ID NO: 33 - protein, 7 aa, Papillomavirus (Human papilloma virus 16)
DRAHYNI

SEQ ID NO: 34 - protein, 9 aa, Papillomavirus (Human papilloma virus 16)
RAHYNIVTF

SEQ ID NO: 35 - protein, 5 aa, Papillomavirus (Human papilloma virus 16)
EYMLD

SEQ ID NO: 36 - protein, 4 aa, Papillomavirus (Human papilloma virus 16)
IDGP

SEQ ID NO: 37 - protein, 5 aa, Papillomavirus (Human papilloma virus 16)
QAEPD

SEQ ID NO: 38 - protein, 11 aa, Papillomavirus (Human papilloma virus 18)
VNHQHLPARRA

SEQ ID NO: 39 - protein, 10 aa, Papillomavirus (Human papilloma virus 18)
DDLRAFQQLF

SEQ ID NO: 40 - protein, 5 aa, Rhinovirus (Human rhinovirus)
SANEV

SEQ ID NO: 41 - protein, 7 aa, Rhinovirus (Human rhinovirus)
NANRQNE

SEQ ID NO: 42 - protein, 5 aa, Rhinovirus (Human rhinovirus)
DNHRE

SEQ ID NO: 43 - protein, 16 aa, Rhinovirus (Human rhinovirus)
NTEPVIKKRKGDIKSY

SEQ ID NO: 44 - DNA, 2628 bp, Rhinovirus/Influenzavirus A,B chimera
atgggcgctcaggtttctacacagaaaagtggatctcacgaaaatcaaaacattttgaccaatggatcaaatcagactttcacagttataaattactataaggatgcagcaagtacatcatcagctggtcaatcactgtcaatggacccatctaagtttacagaaccagttaaagatctcatgcttaagggtgcaccagcattgaattcacccaatgttgaggcctgtggttatagtgatagagtacaacaaatcacactcgggaattcaacaataacaacacaagaagcagccaacgctgttgtgtgttatgctgaatggccagagtaccttccagatgtggacgctagtgatgtcaataaaacttcaaaaccagacacttctgtctgtaggttttacacattggatagtaagacatggacaacaggttctaaaggctggtgctggaaattaccagatgcactcaaagatatgggtgtgttcgggcaaaacatgtttttccactcactaggaagatcaggttacacagtacacgttcagtgcaatgccacaaaattccatagcggttgtctacttgtagttgtaataccagaacaccaactggcttcacatgagggtggcaatgtttcagttaaatacacattcacgcatccaggtgaacgtggtatagatttatcatctgcacagcccgcatcattattaacagaagttgaaacaccaataagaaatgaatggggctcgagaaatgaagtgggagggcctgtcaaggatgtcatatacaatatgaatggtactttattaggaaatctgctcattttccctcaccagttcattaatctaagaaccaataatacagccacaatagtgataccatacataaactcagtacccattgattcaatgacacgtcacaacaatgtctcactgatggtcatccctattgcccctcttacagtaccaactggagcaactccctcactccctataacagtcacaatagcacctatgtgcactgagttctctgggataaggtccaagtcaattgtgccacaaggtttgccaactacaactttgccggggtcaggacaattcttgaccacagatgacaggcaatcccccagtgcactgccaaattatgagccaactccaagaatacacataccagggaaagttcataacttgctagaaattatacaggtagatacactcattcctatgaacaacacgcatacaaaagatgaggttaacagttacctcataccactaaatgcaaacaggcaaaatgagcaggtttttgggacaaacctgtttattggtgatggggtcttcaaaactactcttctgggtgaaattgttcagtactatacacattggtctggatcacttagattctctttgatgtatactggtcctgccttgtccagtgctaaactcattctagcatacaccccgcctggtgctcgtggtccacaggacaggagagaagcaatgctaggtactcatgttgtctgggatattggtctgcaatccaccatagtaatgacaataccatggacatcaggggtgcagtttagatatactgatccagatacatacaccagtgctggctttctatcatgttggtatcaaacttctcttatacttcccccagaaacgaccggccaggtctacttattatcattcataagtgcatgtccagattttaagcttaggctgatgaaagatactcaaactatctcacagactgttgcactcactgaaggcttaggtgatgaattagaagaagtcatcgttgagaaaacgaaacagacggtggcctcaatctcatctggtccaaaacacacacaaaaagtccccatactaactgcaaacgaaacaggggccacaatgcctgttcttccatcagacagcatagaaaccagaactacctacatgcactttaatggttcagaaactgatgtagaatgctttttgggtcgtgcagcttgtgtgcatgtaactgaaatacaaaacaaagatgctactggaatagataatcacagagaagcaaaattgttcaatgattggaaaatcaacctgtccagccttgtccaacttagaaagaaactagaactcttcacttatgttaggtttgattctgagtataccatactggccactgcatctcaacctgattcagcaaactattcaagcaatttggtggtccaagccatgtatgttccacctggtgccccgaatccaaaagagtgggacgattacacatggcaaagtgcttcaaaccccagtgtattcttcaaggtgggggatacatccaggtttagtgtgccttatgtaggattggcatcagcatataattgtttttatgatggttactcacatgatgatgcagaaactcagtatggcataactgttctaaaccatatgggtagtatggcattcagaatagtaaatgaacatgatgaacataaaactcttgtcaagatcagagtttatcacagggcaaagcacgttgaagcatggattccaagagcacccagagcactaccctacacatcaatagggcgcacaaattatcctaagaatacagaaccagtaattaagaagaggaaaggtgacattaaatcctat

SEQ ID NO: 45 - DNA, 2646 bp, Rhinovirus/Influenzavirus A,B chimera
atgggcgctcaggtttctacacagaaaagtggatctcacgaaaatcaaaacattttgaccaatggatcaaatcagactttcacagttataaattactataaggatgcagcaagtacatcatcagctggtcaatcactgtcaatggacccatctaagtttacagaaccagttaaagatctcatgcttaagggtgcaccagcattgaattcacccaatgttgaggcctgtggttatagtgatagagtacaacaaatcacactcgggaattcaacaataacaacacaagaagcagccaacgctgttgtgtgttatgctgaatggccagagtaccttccagatgtggacgctagtgatgtcaataaaacttcaaaaccagacacttctgtctgtaggttttacacattggatagtaagacatggacaacaggttctaaaggctggtgctggaaattaccagatgcactcaaagatatgggtgtgttcgggcaaaacatgtttttccactcactaggaagatcaggttacacagtacacgttcagtgcaatgccacaaaattccatagcggttgtctacttgtagttgtaataccagaacaccaactggcttcacatgagggtggcaatgtttcagttaaatacacattcacgcatccaggtgaacgtggtatagatttatcatctgcaggcacccactcattattaaaagaagttgaaacaccaataagaaatgaatggggctcgagatcaaatgattcatcagataatgaagtgggagggcctgtcaaggatgtcatatacaatatgaatggtactttattaggaaatctgctcattttccctcaccagttcattaatctaagaaccaataatacagccacaatagtgataccatacataaactcagtacccattgattcaatgacacgtcacaacaatgtctcactgatggtcatccctattgcccctcttacagtaccaactggagcaactccctcactccctataacagtcacaatagcacctatgtgcactgagttctctgggataaggtccaagtcaattgtgccacaaggtttgccaactacaactttgccggggtcaggacaattcttgaccacagatgacaggcaatcccccagtgcactgccaaattatgagccaactccaagaatacacataccagggaaagttcataacttgctagaaattatacaggtagatacactcattcctatgaacaacacgcatacaaaagatgaggttaacagttacctcataccactaaatgcaaacaggcaaaatgagcaggtttttgggacaaacctgtttattggtgatggggtcttcaaaactactcttctgggtgaaattgttcagtactatacacattggtctggatcacttagattctctttgatgtatactggtcctgccttgtccagtgctaaactcattctagcatacaccccgcctggtgctcgtggtccacaggacaggagagaagcaatgctaggtactcatgttgtctgggatattggtctgcaatccaccatagtaatgacaataccatggacatcaggggtgcagtttagatatactgatccagatacatacaccagtgctggctttctatcatgttggtatcaaacttctcttatacttcccccagaaacgaccggccaggtctacttattatcattcataagtgcatgtccagattttaagcttaggctgatgaaagatactcaaactatctcacagactgttgcactcactgaaggcttaggtgatgaattagaagaagtcatcgttgagaaaacgaaacagacggtggcctcaatctcatctggtccaaaacacacacaaaaagtccccatactaactgcaaacgaaacaggggccacaatgcctgttcttccatcagacagcatagaaaccagaactacctacatgcactttaatggttcagaaactgatgtagaatgctttttgggtcgtgcagcttgtgtgcatgtaactgaaatacaaaacaaagatgctactggaatagataatcacagagaagcaaaattgttcaatgattggaaaatcaacctgtccagccttgtccaacttagaaagaaactagaactcttcacttatgttaggtttgattctgagtataccatactggccactgcatctcaacctgattcagcaaactattcaagcaatttggtggtccaagccatgtatgttccacctggtgccccgaatccaaaagagtgggacgattacacatggcaaagtgcttcaaaccccagtgtattcttcaaggtgggggatacatccaggtttagtgtgccttatgtaggattggcatcagcatataattgtttttatgatggttactcacatgatgatgcagaaactcagtatggcataactgttctaaaccatatgggtagtatggcattcagaatagtaaatgaacatgatgaacataaaactcttgtcaagatcagagtttatcacagggcaaagcacgttgaagcatggattccaagagcacccagagcactaccctacacatcaatagggcgcacaaattatcctaagaatacagaaccagtaattaagaagaggaaaggtgacattaaatcctat

SEQ ID NO: 46 - protein, 23 aa, Influenzavirus A,B (Human influenza virus)
SLLTEVETPIRNEWGCRCNGSSD

SEQ ID NO: 47 - protein, 23 aa, Influenzavirus A,B (Human influenza virus)
SLLTEVETPTKNEWECRCNDSSD

SEQ ID NO: 48 - protein, 23 aa, Influenzavirus A,B (Human influenza virus)
SLLTEVETPIRNEWECRCNGSSD

SEQ ID NO: 49 - protein, 23 aa, Influenzavirus A,B (Human influenza virus)
SLLTEVETPIRNEWECRCNDSSD

SEQ ID NO: 50 - DNA, 7212 bp, Rhinovirus (Human rhinovirus)
ttaaaacagcggatgggtatcccaccattcgacccattgggtgtagtactctggtactatgtacctttgtacgcctgtttctccccaaccacccttccttaaaattcccacccatgaaacgttagaagcttgacattaaagtacaataggtggcgccatatccaatggtgtctatgtacaagcacttctgtttccccggagcgaggtataggctgtacccactgccaaaagcctttaaccgttatccgccaaccaactacgtaacagttagtaccatcttgttcttgactggacgttcgatcaggtggattttccctccactagtttggtcgatgaggctaggaattccccacgggtgaccgtgtcctagcctgcgtggcggccaacccagcttatgctgggacgcccttttaaggacatggtgtgaagactcgcatgtgcttggttgtgagtcctccggcccctgaatgcggctaaccttaaccctggagccttatgccacgatccagtggttgtaaggtcgtaatgagcaactccgggacgggaccgactactttgggtgtccgtgtttctcatttttcttcatattgtcttatggtcacagcatatatatacatatactgtgatcatgggcgctcaggtttctacacagaaaagtggatctcacgaaaatcaaaacattttgaccaatggatcaaatcagactttcacagttataaattactataaggatgcagcaagtacatcatcagctggtcaatcactgtcaatggacccatctaagtttacagaaccagttaaagatctcatgcttaagggtgcaccagcattgaattcacccaatgttgaggcctgtggttatagtgatagagtacaacaaatcacactcgggaattcaacaataacaacacaagaagcagccaacgctgttgtgtgttatgctgaatggccagagtaccttccagatgtggacgctagtgatgtcaataaaacttcaaaaccagacacttctgtctgtaggttttacacattggatagtaagacatggacaacaggttctaaaggctggtgctggaaattaccagatgcactcaaagatatgggtgtgttcgggcaaaacatgtttttccactcactaggaagatcaggttacacagtacacgttcagtgcaatgccacaaaattccatagcggttgtctacttgtagttgtaataccagaacaccaactggcttcacatgagggtggcaatgtttcagttaaatacacattcacgcatccaggtgaacgtggtatagatttatcatctgcaaatgaagtgggagggcctgtcaaggatgtcatatacaatatgaatggtactttattaggaaatctgctcattttccctcaccagttcattaatctaagaaccaataatacagccacaatagtgataccatacataaactcagtacccattgattcaatgacacgtcacaacaatgtctcactgatggtcatccctattgcccctcttacagtaccaactggagcaactccctcactccctataacagtcacaatagcacctatgtgcactgagttctctgggataaggtccaagtcaattgtgccacaaggtttgccaactacaactttgccggggtcaggacaattcttgaccacagatgacaggcaatcccccagtgcactgccaaattatgagccaactccaagaatacacataccagggaaagttcataacttgctagaaattatacaggtagatacactcattcctatgaacaacacgcatacaaaagatgaggttaacagttacctcataccactaaatgcaaacaggcaaaatgagcaggtttttgggacaaacctgtttattggtgatggggtcttcaaaactactcttctgggtgaaattgttcagtactatacacattggtctggatcacttagattctctttgatgtatactggtcctgccttgtccagtgctaaactcattctagcatacaccccgcctggtgctcgtggtccacaggacaggagagaagcaatgctaggtactcatgttgtctgggatattggtctgcaatccaccatagtaatgacaataccatggacatcaggggtgcagtttagatatactgatccagatacatacaccagtgctggctttctatcatgttggtatcaaacttctcttatacttcccccagaaacgaccggccaggtctacttattatcattcataagtgcatgtccagattttaagcttaggctgatgaaagatactcaaactatctcacagactgttgcactcactgaaggcttaggtgatgaattagaagaagtcatcgttgagaaaacgaaacagacggtggcctcaatctcatctggtccaaaacacacacaaaaagtccccatactaactgcaaacgaaacaggggccacaatgcctgttcttccatcagacagcatagaaaccagaactacctacatgcactttaatggttcagaaactgatgtagaatgctttttgggtcgtgcagcttgtgtgcatgtaactgaaatacaaaacaaagatgctactggaatagataatcacagagaagcaaaattgttcaatgattggaaaatcaacctgtccagccttgtccaacttagaaagaaactagaactcttcacttatgttaggtttgattctgagtataccatactggccactgcatctcaacctgattcagcaaactattcaagcaatttggtggtccaagccatgtatgttccacctggtgccccgaatccaaaagagtgggacgattacacatggcaaagtgcttcaaaccccagtgtattcttcaaggtgggggatacatccaggtttagtgtgccttatgtaggattggcatcagcatataattgtttttatgatggttactcacatgatgatgcagaaactcagtatggcataactgttctaaaccatatgggtagtatggcattcagaatagtaaatgaacatgatgaacataaaactcttgtcaagatcagagtttatcacagggcaaagcacgttgaagcatggattccaagagcacccagagcactaccctacacatcaatagggcgcacaaattatcctaagaatacagaaccagtaattaagaagaggaaaggtgacattaaatcctatggtttaggacctaggtacggtgggatttatacatcaaatgttaaaataatgaattaccacttgatgacaccagaagaccaccataatctgatagcaccctatccaaatagagatttagcaatagtctcaacaggaggacatggtgcagaaacaataccacactgtaactgtacatcaggtgtttactattccacatattacagaaagtattaccccataatttgtgaaaagcccaccaacatctggattgaaggaaacccttattacccaagtaggtttcaagcaggagtgatgaaaggggttgggccagcagaaccaggagactgcggtgggattttgagatgcatacatggtcccattggattgttaacagctggaggtagtggatatgtttgttttgctgacatacgacagttggagtgtatcgcagaggaacaggggctgagtgattacatcacaggtttgggtagagcttttggtgtcgggttcactgaccaaatctcaacaaaagtcacagaactacaagaagtggcgaaagatttcctcaccacaaaagttttgtccaaagtggtcaaaatggtttcagctttagtgatcatttgcagaaatcatgatgacttggtcactgttacggccactctagcactacttggatgtgatggatctccctggagatttctgaagatgtacatttccaaacactttcaggtgccttacattgaaagacaagcaaatgatggatggttcagaaagtttaatgatgcatgtaatgctgcaaagggattggaatggattgctaataagatttccaaactgattgaatggataaaaaacaaagtacttccccaagccaaagaaaaactagaattttgtagtaaactcaaacaacttgatatactagagagacaaataaccaccatgcatatctcgaatccaacacaggaaaaacgagagcagttgttcaacaacgtattgtggttggaacaaatgtcgcaaaagtttgccccacattatgccgttgaatcaaaaagaatcagggaactcaagaacaaaatggtaaattatatgcaatttaaaagtaaacaaagaactgaaccagtgtgtgtattaatccatggtacacccggttctggtaaatcattaacaacatccattgtgggacgtgcaattgcagaacacttcaattcagcagtatattcacttccaccagatcccaagcactttgatggttatcagcaacaggaagttgtgattatggatgatctgaaccaaaatccagatggacaggatataagcatgttttgtcaaatggtttcttcagtggatttcttgcctccaatggctagtttagataacaagggcatgttattcaccagtaattttgttctagcctccacaaattctaacacactaagccccccaacaatcttgaatcctgaagctttagtcaggagatttggttttgacctggatatatgtttgcatactacctacacaaagaatggaaaactcaatgcaggcatgtcaaccaagacatgcaaagattgccatcaaccatctaatttcaagaaatgttgccccctggtctgtggaaaagctattagcttggtagacagaactaccaacgttaggtatagtgtggatcaactggtcacagctattataagtgatttcaagagcaaaatgcaaattacagattccctagaaacactgtttcaaggaccagtgtataaagatttagagattgatgtttgcaacacaccacctccagaatgtatcaacgatttactgaaatctgtagattcagaagagattagggaatattgtaagaagaagaaatggattatacctgaaattcctaccaacatagaaagggctatgaatcaagccagcatgattattaatactattctgatgtttgtcagtacattaggtattgtttatgtcatttataaattgtttgctcaaactcaaggaccatattctggtaacccgcctcacaataaactaaaagccccaactttacgcccagttgttgtgcaaggaccaaacacagaatttgcactatccctgttaaggaaaaacataatgactataacaacctcaaagggagagttcacagggttaggcatacatgatcgtgtctgtgtgatacccacacacgcacagcctggtgatgatgtactagtgaatggtcagaaaattagagttaaggataagtacaaattagtagatccagagaacattaatctagagcttacagtgttgactttagatagaaatgaaaaattcagagatatcaggggatttatatcagaagatctagaaggtgtggatgccactttggtagtacattcaaataactttaccaacactatcttagaagttggccctgtaacaatggcaggacttattaatttgagtagcacccccactaacagaatgattcgttatgattatgcaacaaaaactgggcagtgtggaggtgtgctgtgtgctactggtaagatctttggtattcatgttggcggtaatggaagacaaggattttcagctcaacttaaaaaacaatattttgtagagaaacaaggccaagtaatagctagacataaggttagggagtttaacataaatccagtcaacacgccaaccaagtcaaaattacatcccagtgtattctatgatgttttcccaggtgacaaggaacctgctgtattgagtgacaatgatcccagactggaagttaaattgactgaatcattattctctaagtacaaggggaatgtaaatacggaacccactgaaaatatgcttgtggctgtagaccattatgcagggcaactattatcactagatatccccacttctgaacttacactaaaagaagcattatatggagtagatggactagaacctatagatattacaaccagtgcaggatttccctatgtgagtcttgggatcaaaaagagagacattctgaacaaagagacccaggacacagaaaagatgaagttttatctagacaagtatggcattgacttgcctctagttacatatattaaggatgaattaagaagtgttgacaaagtccgattagggaaaagtagattaattgaagcctccagtttgaatgattctgttaacatgagaatgaaactaggcaacctttacaaagcattccatcaaaatcccggtgttctgactgggtcagcagtgggttgtgatcctgatgtgttttggtctgtcatcccttgcttaatggatgggcacctgatggcatttgattactctaattttgatgcctctttgtcaccagtttggtttgtctgtctagagaaggttttgaccaagttaggctttgcaggctcttcattaattcaatcaatttgtaatacccatcatatctttagggatgaaatatatgtggttgaaggtggcatgccctcagggtgttcaggaaccagcatattcaattccatgatcaacaacataatcattaggactttgatattagatgcatataaaggaatagatttagacaaacttaaaatcttagcttacggtgatgatttgattgtttcttatccttatgaactggatccacaagtgttggcaactcttggtaaaaattatggactaaccatcacacccccagacaaatctgaaacttttacaaaaatgacatgggaaaacttgacatttttaaagagatacttcaagcctgatcaacaatttccctttttggttcacccagttatgcccatgaaagatatacatgagtcaatcagatggacaaaggatcctaaaaacacacaggatcacgtccgatcattatgcatgttagcatggcactcaggagaaaaagagtacaatgaattcattcagaagatcagaactactgacattggaaaatgtctaattctcccagaatacagcgtacttaggaggcgctggttggacctcttttaggttaacaatatagacacttaatttgagtagaagtaggagtttat

SEQ ID NO: 51 - protein, 24 aa, Influenzavirus A,B (Avian influenza virus)
MSLLTEVETPTRNGWECRCSDSSD

SEQ ID NO: 52 - protein, 24 aa, Influenzavirus A,B (Avian influenza virus)
MSLLTEVETPTRNGWECKCSDSSD

SEQ ID NO: 53 - protein, 24 aa, Influenzavirus A,B (Avian influenza virus)
MSLLTEVETHTRNGWGCRCSDSSD

SEQ ID NO: 54 - protein, 24 aa, Influenzavirus A,B (Human influenza virus)
MSLLTEVETLTRNGWECKCSDSSD

(19) United States
(12) Patent Application Publication
(10) Pub. No.: US 2010/0239605 A1
(43) Pub. Date: Sep. 23, 2010

(54) Recombinant Rhinovirus Vectors

(75) Inventors: Kirill Kalnin, Pelham, NH (US); Yanhua Yan, Westford, MA (US); Harold Kleanthous, Westford, MA (US)

Correspondence Address: CLARK & ELBING LLP, 101 FEDERAL STREET, BOSTON, MA, 02110, US

(73) Assignee: Sanofi Pasteur Biologics Co., Cambridge, MA (US)

(21) Appl. No.: 12/442,990

(22) PCT Filed: Oct. 1, 2007
(86) PCT No.: PCT/US07/21102

§ 371 Date: May 17, 2010

Related U.S. Application Data
(60) Provisional application No. 60/880,664, filed on Jan. 15, 2007.
(60) Provisional application No. 60/848,308, filed on Sep. 29, 2006.

(51) Int. Cl. A61K 39/12; C12N 7/01; A61K 35/76; C07K 14/11; C40B 30/00; A61P 37/04; A61P 31/16

(52) U.S. Cl. 424/199.1; 435/235.1; 424/93.2; 530/350; 506/7

(57) ABSTRACT

The invention provides recombinant rhinovirus vectors including, for example, influenza virus antigens. Also provided by the invention are corresponding pharmaceutical compositions and methods.

### BACKGROUND OF THE INVENTION

An influenza pandemic occurs when a new influenza virus subtype appears, against which the global population has little or no immunity. During the 20^{th} century, influenza pandemics caused millions of deaths, social disruption, and profound economic losses worldwide. Influenza experts agree that another pandemic is likely to happen, but it is unknown when. The level of global preparedness at the moment when a pandemic strikes will determine the public health and economic impact of the disease. As of today, the World Health Organization (WHO) estimates that there will be at least several hundred million outpatient visits, more than 25 million hospital admissions, and several million deaths globally, within a very short period. These concerns were highlighted in 2003, when the avian H5N1 virus reached epizootic levels in domestic fowl in a number of Asian countries, and then spread to Europe and Africa. Fortunately, its transmission to humans has so far been limited, with 246 documented infections, which were associated with high mortality accounting for 144 deaths (Sep. 14, 2006; World Health Organization (WHO) Web site).

Conventional influenza vaccines are designed to elicit neutralizing antibody responses against influenza virus hemagglutinin protein (HA). Due to the constant antigenic drift in the HA protein, the vaccine composition must be changed each year to match anticipated circulating viral strains. Such a vaccine approach is unacceptable in the face of a pandemic, because of the long time required for the isolation and identification of a pandemic strain, and construction and manufacture of an appropriate vaccine. A more effective approach to control or prevention of an influenza pandemic contemplates development of a “universal” vaccine capable of eliciting protective immunity against recently identified, highly conserved influenza virus immunological determinants. Such a vaccine should provide broad protection across influenza A virus strains. Further, such a vaccine could be manufactured throughout the year, stockpiled, and/or administered throughout the year.

The influenza matrix protein M2 has been demonstrated to serve as an effective target for vaccine development (DeFilette et al., Virology 337:149-161, 2005). M2 is a 97-amino-acid transmembrane protein of influenza type A virus (Lamb et al., Proc. Natl. Acad. Sci. U.S.A 78:4170-4174, 1981; Lamb et al., Cell 40:627-633, 1985). The mature protein forms homotetramers (Holsinger et al., Virology 183:32-43, 1991; Sugrue et al., Virology 180:617-624, 1991) that have pH-inducible ion channel activity (Pinto et al., Cell 69:517-528, 1992; Sugrue et al., Virology 180:617-624, 1991). M2-tetramers are expressed at high density in the plasma membrane of infected cells and are also incorporated at low frequency into the membranes of mature virus particles (Takeda et al., Proc. Natl. Acad. Sci. U.S.A. 100:14610-14617, 2003; Zebedee et al., J. Virol. 62:2762-2772, 1998). The M2 N-terminal 24-amino-acid ectodomain (M2e) is highly conserved among type A influenza viruses (Piers et al., Virus Res. 103:173-176, 2004). The high degree of conservation of M2e can be explained by constraints resulting from its genetic relationship with M1, the most conserved protein of the virus (Ito et al., J. Virol. 65:5491-5498, 1991), and the absence of M2e specific antibodies during natural infection (Black et al., J. Gen. Virol. 74 (Pt. 1):143-146, 1993).

As shown in the alignment below, obtained using sequences from the NCBI influenza database (http://www.ncbi.nlm.nih.gov/genomes/FLU/Database/multiple.cgi), avian H5N1 influenza virus M2e appears to be evolving toward the consensus sequence found in typical human H1, H2, and H3 viruses, suggesting that broad protection, including from new avian viruses, using the “human” influenza M2e epitope may be a possibility:

| | |
|---|---|
| Human H1N1 (SEQ ID NO: 1) | MSLLTEVETPIRNEWGCRCNDSSD |
| Human H5N1 2001-2006 | ..........T....E...S.... |
| (SEQ ID NO: 5) | |
| Human H5N1 1997-2000 | .........LT..G.....S.... |
| (SEQ ID NO: 6) | |
| Avian H5N1 1983-1998 | .........LT..G.....S.... |
| (SEQ ID NO: 6) | |

The phenomenon of evolution of the H5N1 M2e towards the H1N1 M2e sequence was recently reported based on the analysis of sequences of 800 H5H1 strains isolated from humans and birds in Indonesia and Vietnam (Smith et al., Virology 350:258-268, 2006). The evolved avian M2e peptide EVETPTRN (SEQ ID NO:2), but not its “predecessor” EVETLTRN (SEQ ID NO:3), was efficiently recognized by an anti-human M2e monoclonal antibody (Mab)(Liu et al., Microbes. Infect. 7:171-177, 2005). This is important, because some “bird-flu-like” changes have been shown previously to reduce the effectiveness of protection provided by human M2e specific Mabs. Interestingly, some “bird-flu-like” amino acid changes in M2e reduced pathogenicity of human H1N1 viruses in mice (Zharikova et al., J. Virol. 79:6644-6654, 2005).

The WHO has emphasized the possibility of “simultaneous occurrence of events with pandemic potential with different threat levels in different countries, as was the case in 2004 with poultry outbreaks of H7N3 in Canada and H5N1 in Asia” (http://www.who.int/en/). As is shown in the alignment below, M2e H7N7 differs at only one amino acid from the “humanized” variant of H5N1. The H7N7 subtype has demonstrated the ability to be transmissible between species (Koopmans et al., Lancet 363:587-593, 2004) and can be lethal for people (Fouchier et al., Proc. Natl. Acad. Sci. U.S.A 101:1356-1361, 2004). The other strains (H9N2) were also shown to be able to infect poultry and spread to people (Cameron et al., Virology 278:36-41, 2000; Li et al., J. Virol. 77:6988-6994, 2003; Wong et al., Chest 129:156-168, 2006).

| | |
|---|---|
| Human H1N1 (SEQ ID NO: 1) | MSLLTEVETPIRNEWGCRCNDSSD |
| Avian/Equine H7N7 | ..........T..G.E...S.... |
| (SEQ ID NO: 51) | |
| Avian H9Nx 1966-1996 | ..........T..G.E.K.S.... |
| (SEQ ID NO: 52) | |
| Avian H9Nx 1997-2004 | .........HT..G.....S.... |
| (SEQ ID NO: 53) | |
| Human H9N2 1999-2003 | .........LT..G.E.K.S.... |
| (SEQ ID NO: 54) | |

M2e-based recombinant protein vaccines have been shown to elicit protective immune responses against both homologous and heterologous influenza A virus challenge (Fiers et al., Virus Res. 103:173-176, 2004; Slepushkin et al., Vaccine 13:1399-1402, 1995). More recent studies using an M2e peptide conjugated to keyhole limpet hemocyanin and N. meningitides outer membrane protein illustrated good immune responses not only in mice, but also in ferrets and rhesus monkeys (Fan et al., Vaccine 22:2993-3003, 2004). Protection against H1, H5, H6, and H9 influenza A viruses with a liposomal M2e vaccine was demonstrated in mice recently (Fan et al., Vaccine 22:2993-3003, 2004).

Development of delivery systems for influenza antigens is important for the development of vaccines against influenza virus infection, such as pandemic vaccines.

### SUMMARY OF THE INVENTION

The invention provides, in a first aspect, rhinovirus vectors that include antigens, as described herein, such as influenza virus antigens (e.g., M2e peptides). The vectors can be non-pathogenic in humans (e.g., Human Rhinovirus 14 (HRV14). The antigens can be inserted into the vectors of the invention at, for example, the site of a neutralizing immunogen selected from the group consisting of Neutralizing Immunogen I (NimI), Neutralizing Immunogen II (NimII) (e.g., between amino acids 158 and 160 of NimII), Neutralizing Immunogen III (NimIII), and Neutralizing Immunogen IV (NimIV), or a combination thereof. The antigen (e.g., influenza virus antigen) optionally can be flanked by linker sequences on one or both ends. The rhinovirus vectors of the invention can be live or inactivated.

In a second aspect, the invention provides pharmaceutical compositions that include the rhinovirus vectors described herein and one or more pharmaceutically acceptable carriers or diluents. Optionally, such pharmaceutical compositions can further include an adjuvant (e.g., aluminum or chitin-based adjuvants), and/or one or more additional active ingredients (e.g., a Hepatitis B core protein fused with an antigen sequence, such as an M2e sequence).

In a third aspect, the invention provides methods of inducing an immune response to an antigen (e.g., an influenza virus antigen) in a subject (e.g., a human subject), involving administering to the subject a pharmaceutical composition as described herein. In one example, the subject does not have but is at risk of developing an infection, such as an influenza virus infection. In another example, the subject has an infection to which the vector induces immunity, such as an influenza virus infection. In various examples, the pharmaceutical composition is administered to the subject intranasally.

In a fourth aspect, the invention provides methods of making pharmaceutical compositions as described herein, involving admixing a rhinovirus vector as described herein and one or more pharmaceutically acceptable carriers or diluents. Optionally, these methods can involve addition of adjuvants, reconstitution of lyophilized materials, and/or admixture with other active ingredients.

In a fifth aspect, the invention provides nucleic acid molecules encoding or corresponding to the genome of the rhinovirus vectors described herein.

In a sixth aspect, the invention provides Nimll peptides including one or more heterologous antigen sequences, such as an inserted influenza virus antigen sequence (e.g., an M2e sequence).

In a seventh aspect, the invention provides methods of generating rhinovirus vectors as described herein, including an antigen, such as an influenza virus antigen (e.g., influenza virus M2e). These methods can include the steps of (i) generating a library of recombinant rhinovirus vectors based on an infectious cDNA clone that includes inserted antigen sequences (e.g., influenza virus antigen sequences), and (ii) selecting from the library recombinant viruses that (a) maintain inserted sequences upon passage, and (b) are neutralized with antibodies against the inserted sequence. In one example of these methods; the rhinovirus vector is human rhinovirus 14 (HRV14). In other examples, the inserted antigen sequence is inserted at a position selected from the group consisting of NimI, NimII, NimIII, and NimIV Optionally, the inserted antigen sequence is flanked on one or both ends with random linker sequences, as described herein.

In an eighth aspect, the invention provides methods of cultivating rhinovirus vectors including inserted antigen (e.g., influenza virus antigen) sequences. These methods involve the passaging the vectors in HeLa or MRC-5 cells.

The invention provides several advantages. For example, use of a live vector system to deliver antigens such as M2e provides advantages including: (i) the ability to elicit very strong and long-lasting antibody responses with as little as a single dose of vaccine, and (ii) greater scalability of manufacturing (i.e., more doses at a lower cost) when compared with subunit or killed vaccines. Thus, in a pandemic situation, many more people could be immunized in a relatively short period of time with a live vaccine. In addition, the HRV vectors of the invention can be delivered intranasally, resulting in both systemic and mucosal immune responses. Use of HRV14 provides additional advantages, as it is nonpathogenic and is infrequently observed in human populations (Andries et al., J. Virol. 64:1117-1123, 1990; Lee et al., Virus Genes 9:177-181, 1995), which reduces the probability of preexisting anti-vector immunity in vaccine recipient. Further, the amount of HRV needed to infect humans is very small (one tissue culture infectious dose (TCID₅₀) (Savolainen-Kopra, “Molecular Epidemiology of Human Rhinoviruses,” Publications of the National Public Health Institute 2/2006, Helsinki, Finland, 2006)), which is a favorable feature in terms of cost-effectiveness of HRV-based vaccine manufacturing.

Other features and advantages of the invention will be apparent from the following Detailed Description, the Drawings, and the Claims.

### BRIEF DESCRIPTION OF THE DRAWINGS

FIG. 1 is a schematic representation of a virus particle (upper panel) and genome (lower panel) of HRV14. The human rhinovirus 14 (HRV14) capsid exhibits a pseudo-T=3 (P=3) isochedral symmetry and consists of 60 copies of viral proteins VP1, VP2, VP3, and VP4, with VP4 at the RNA-capsid interface (Rossmann et al., Nature 317:145-153, 1985). VP1-3 proteins form a canyon containing a receptor-binding site for a cellular receptor, intracellular adhesion molecule 1 (ICAM-1) (Colonno et al., J. Virol. 63:36-42, 1989). Three major neutralizing immunogenic (Nim) sites, NimI(AB), NimII, and NimIII, were identified on the surface of the canyon rim as binding sites for neutralizing antibodies (Sherry et al., J. Virol. 57:246-257, 1986). The reconstruction of the HRV14 particle was created in Chimera program on the basis of HRV14 crystal structure with NimI-specific mAb 17 (protein databank database #1RVF).

FIG. 2 is described as follows: (A) HRV14-M2e constructs created in this study. A derivative of the HRV14 cDNA clone, plasmid pWR1, was used for construction of M2e-insertion mutants (SEQ ID NOS:7-8). (B) Plaques produced by HRV14-NimII-XXX17AA and HRV14-NimII-XXX23AA virus libraries, as well as wild type HRV14 derived from pWR1. Construct #1 did not yield plaques, as discussed in the text and supported by additional data (FIGS. 3 and 4), indicating that the random linker strategy is an effective means of engineering novel epitopes in HRV.

FIG. 3 shows the stability of the M2e insert in different HRV14-M2e constructs. The insert-containing fragments were RT-PCR amplified with pairs of primers, P1-up100Fw, VP 1-dwn200Rv (green), or 14FAflII-1730Rv (red), resulting in “PCR B” (green) or “PCR A” (red) DNA fragments, respectively. These fragments were digested with XhoI. Agarose gel electrophoresis results for HRV14-M2e chimera at passages 2, 3, and 4, and for HRV14-NimII-XXX17AA and HRV14-NimII-XXX17AA virus libraries at passage 4, are shown. The two cleaved fragments (indicated by arrows) represent insert-containing virus.

FIG. 4 shows possible steric interference of the 23 AA M2e insert in the NimII site with the receptor binding domain of HRV14. The insert without linkers could stretch out from NimII and almost reach the opposite side of the canyon (i.e., at the NimI site), as shown in the picture. That barrier could effectively block receptor entrance into the canyon. An N-terminal linker can change position of the insert (direction is shown by arrow) and open access to the canyon. This molecular model of VP1-VP4 subunit of HRV14-NimII-M2e (23 AA) was created in Accelrys Discovery Studio (Accelrys Software, Inc). This illustrates our ability to engineer novel epitopes into HRV14 due to the available structural data and modeling software.

FIG. 5 shows the results of a plaque reduction neutralization test (PRNT) of HRV14, the HRV14-NimII-XXX23AA library, and the HRV14-NimII-XXX17AA library with anti-M2e Mab 14C2 (Abcam, Inc; Cat# ab5416). The results demonstrate efficient neutralization of both libraries, but not of the vector virus (HRV14). The purity of both libraries (absence of WT contamination) is also evident from these results.

FIG. 6 shows M2e-specific IgG antibody response (pooled samples) in immunized mice prior to challenge. End point titers (ETs) are shown after relevant group titles. Time of correspondent immunizations is shown in parentheses (d0 and d21 stand for day 0 and day 21, respectively).

FIG. 7 shows HRV14-specific IgG antibody responses (pooled samples) in immunized mice prior to challenge. (A)-groups immunized with 1, 2, or 3 doses of HRV14-M2e (17AA) virus; (B)-groups immunized with one or two doses of parental HRV14 virus.

FIG. 8 shows individual M2e-specific IgG antibody responses of immunized mice.

FIG. 9 shows M2e-specific antibody isotypes IgG 1 and IgG2a in mice immunized as described in Table 4: (A) IgG1 ELISA (group pooled samples); (B) IgG2a ELISA (group pooled samples); (C) Titles for FIGS. 9A and 9B; (D) Level of M2-e-specific IgG1 (dots) and IgG2a (diamonds) in individual sera samples (dilution 1:2,700) of group 4 (red; first and third sets of data) and group 7 (green; second and fourth sets of data) mice (see Table 4).

FIG. 10 shows M2e-specific antibodies of IgG2a isotype in mice immunized as described in Table 4. (A) ELISA with M2e peptide (group pooled samples); (B) Individual sera samples (dilution 1:2,700) of group 4 (red; first set of data) and group 7 (green; second set of data) mice (see Table 4) tested in ELISA against M2e-specific peptide.

FIG. 11 shows M2e-specific antibodies of IgG2a isotype in mice immunized as described in Table 4 (upper panel).

FIG. 12 shows survival rates of all groups 28 days after challenge with the PR8 Influenza A strain.

FIG. 13 shows morbidity of all groups 28 days after challenge with PR8 Influenza A strain (FIG. 13A); Individual body weights within group 4 (FIG. 13B) and group 7 (FIG. 13C).

FIG. 14 shows M2e-specific IgG antibody response (pooled samples) in immunized mice prior to challenge (for group see Table 5).

FIG. 15 shows the morbidity (percentage of bodyweight) of all groups during 17 days after non-mortal challenge with PR8 Influenza A strain.

FIG. 16 shows the results of plaque reduction neutralization test (PRNT) of HRV14 and HRV6 with mouse anti-HRV14-NimIV^{HRV6} serum. These data served as a proof of immunodominance of NimIV^{HRV6} in the background of HRV14 capsid, suggesting a novel site for insertion of foreign epitopes.

FIG. 17 shows protection of Balb/c mice against lethal intranasal challenge with influenza virus: A) percent survival post-challenge, and B) weight loss post-challenge.

FIG. 18 is a schematic illustration of the insertion sites in the virion proteins of HRV14. M2e can be introduced in the indicated positions of Niml (SEQ ID NO:42), NimII (SEQ ID NO:40), NimIII (SEQ ID NO:41), and NimIV (SEQ ID NO:43). XXXM2e signifies M2e libraries described herein.

FIG. 19 is a schematic representation of the HRV14 structural region, which shows an insertion site within NimII of VP2 as used in two chimeras made according to the invention. The nucleotide sequences of these chimeras, HRV14-M2e (17AA; SEQ ID NO:44) and HRV14-M2e (23AA; SEQ ID NO:45), are also provided.

### DETAILED DESCRIPTION

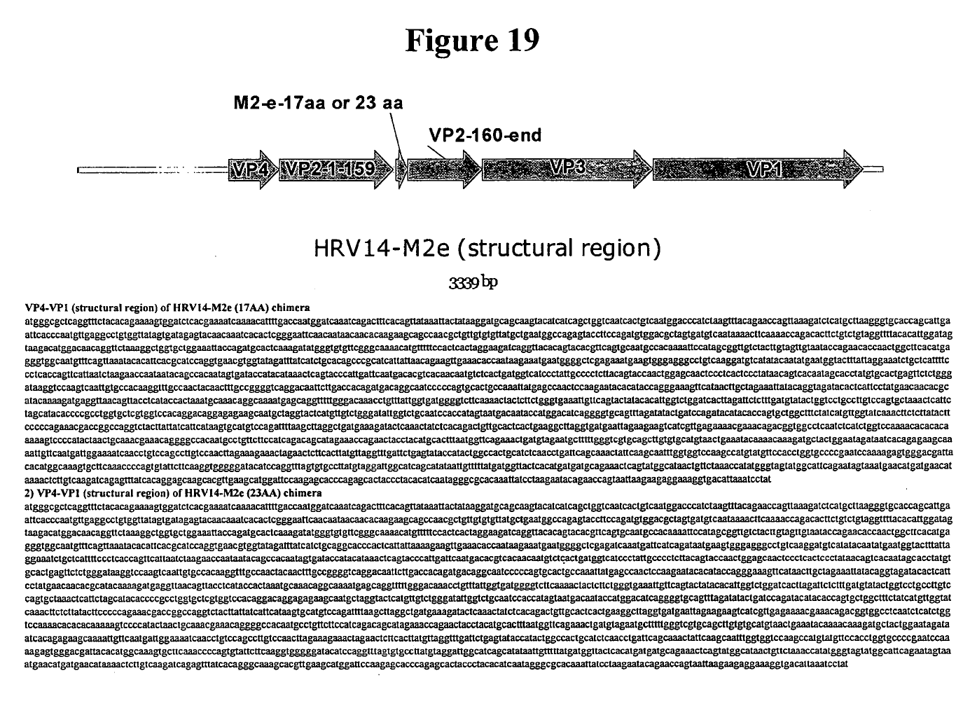

The invention provides universal (pandemic) influenza vaccines, which are based on the use of human rhinoviruses (HRV) as vectors for efficient delivery and presentation of universal influenza virus determinants. As described further below, the extracellular domain of the influenza matrix protein 2 (M2e) is a “universal” epitope that can be included in a universal influenza (influenza A) vaccine, according to the invention. This approach provides an effective influenza pandemic vaccine, which can be administered intranasally to induce local mucosal immunity. Two examples of vaccines according to the invention, HRV14-M2e (17AA) and HRV14-M2e (23AA), are schematically illustrated in FIG. 19, which also includes the nucleotide sequences of these viruses. These are examples of universal influenza vaccine candidates. Based on information such as this, those of skill in the art can now construct vaccine candidates including M2e sequences, as shown in these examples, or other influenza epitopes. Vaccine candidates can also be constructed based on other, non-influenza epitopes, as described further below. The vectors, vaccine compositions, and methods of the invention are described further, as follows.

### HRV Vectors

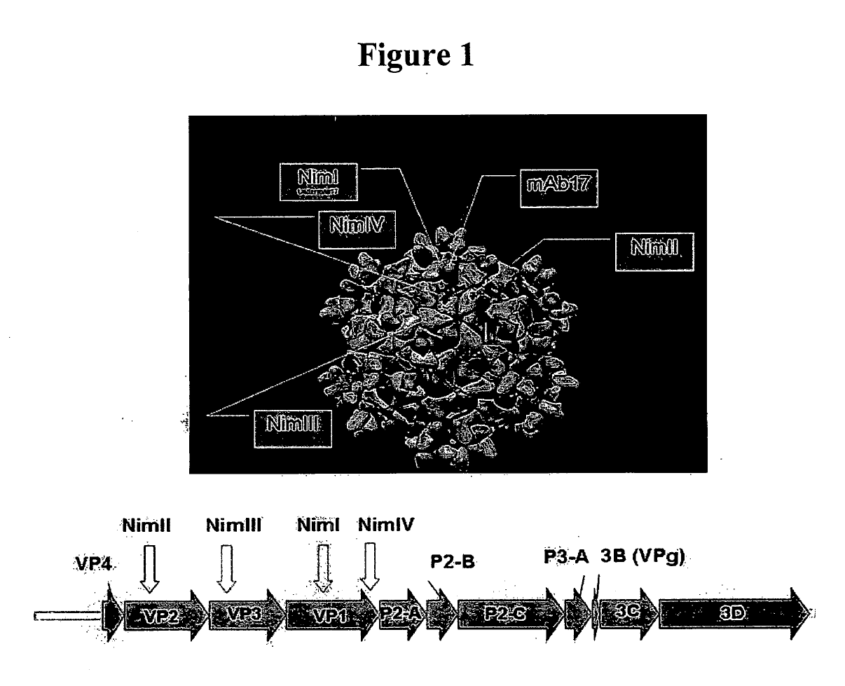

The vectors of the invention are based on human rhinoviruses, such as the non-pathogenic serotype human rhinovirus 14 (HRV14). The HRV14 virus particle and genome structure are schematically illustrated in FIG. 1, which shows virus structural proteins (VP1, VP2, VP3, and VP4), the non-structural proteins (P2-A, P2-B, P-2C, P3-A, 3B(VPg), 3C, and 3D), as well as the locations of major neutralizing immunogenic sites in HRV14 (Nims: NimI, NimII, NimIII, and NimIV).

An example of a molecular clone of HRV14 that can be used in the invention is pWR3.26 (American Type Culture Collection: ATCC® Number: VRMC-7™). This clone is described in further detail below, as well as by Lee et al., J. Virology 67(4):2110-2122, 1993 (also see Sequence Appendix 3). Additional sources of HRV14 can also be used in the invention (e.g., ATCC Accession No. VR284; also see GenBank Accession Nos. L05355 and K02121; Stanway et al., Nucleic Acids Res. 12(20):7859-7875, 1984; and Callahan et al., Proc. Natl. Acad. Sci. U.S.A. 82(3):732-736, 1985). In addition to HRV14, other human rhinovirus serotypes can be used in the invention. As is known in the art, there are more than 100 human rhinovirus serotypes, any of which can be in the invention used upon the derivation of an infectious clone, in the same manner as HRV14. Although described herein with respect to HRV14, the invention applies to other rhinovirus serotypes as well.

Antigen sequences can be inserted into HRV vectors, according to the invention, at different sites, as described further below. In one example, the sequences are inserted into the NimII site of a serotype such as HRV14. NimII (Neutralizing Immunogen II) is an immunodominant region in HRV14 that includes amino acid 210 of VP1 and amino acids 156, 158, 159, 161, and 162 of VP2 (Savolainen-Kopra, “Molecular Epidemiology of Human Rhinoviruses,” Publications of the National Public Health Institute 2/2006, Helsinki, Finland, 2006). In a specific example described below, the sequences are inserted between amino acids 158 and 160 of VP2. Insertions can be made at other sites within the NimII epitope as well. For example, the insertion can be made at any of positions 156, 158, 159, 161, or 162 of VP2, or at position 210 of VP1, or combinations thereof.

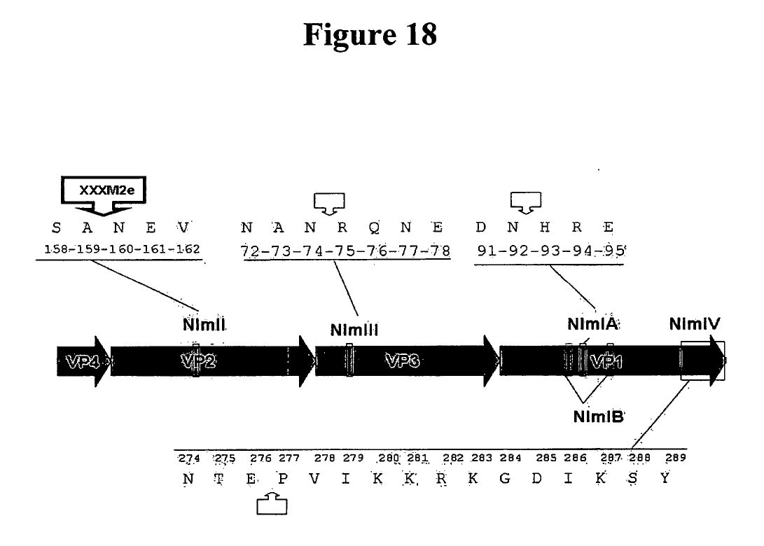

Additional sites at which insertions can be made, alone or in combination with insertions at other sites (e.g., the NimII site), include NimI (A and B), NimIII, and NimIV. Thus, insertions can be made, for example, at positions 91 and/or 95 of VP1 (NimIA), positions 83, 85, 138, and/or 139 of VP1 (NimIB), and/or position 287 of VP1 (NimIII) (see, e.g., FIG. 18). NimIV is in the carboxyl-terminal region of VP1, in a region comprising the following sequence, which represents amino acids 274-289 of HRV14 VP1: NTEPVIKKRKGDIKSY (SEQ ID NO:4). Insertions between any amino acids in this region are included in the invention. Thus, the invention includes, for example, insertions between amino acids 274 and 275; 275 and 276; 276 and 277; 277 and 278; 278 and 279; 279 and 280; 280 and 281; 281 and 282; 282 and 283; 283 and 284; 284 and 285; 285 and 286; 286 and 287; 287 and 288; and 288 and 289. In addition to these insertions, the invention includes insertions where one or more (e.g., 3, 4, 5, 6, 7, 8, 9, or 10) amino acids in this region are deleted. Thus, for example, the invention includes insertions between amino acids 274 and 276; 275 and 277; 276 and 278; 277 and 279; 278 and 280; 279 and 281; 280 and 282; 281 and 283; 282 and 284; 283 and 285; 284 and 286; 285 and 287; 286 and 288; 287 and 289; 288 and 290; and 289 and 291.

The vectors of the invention are made using standard methods of molecular biology, which are exemplified below in the case of a vector including insertions in NimII of HRV14. In addition, and as is discussed further below, the vectors of the invention can be administered in the form of live viruses or can be inactivated prior to administration by, for example, formalin inactivation or ultraviolet treatment, using methods known to those skilled in the art.

Optionally, the vectors may include linker sequences between the HRV vector sequences and the inserted influenza sequences, on the amino and/or carboxyl-terminal ends. These linker sequences can be used to provide flexibility to inserted sequences, enabling the inserted sequences to present the inserted epitope in a manner in which it can induce an immune response. Examples of such linker sequences are provided below. Identification of linker sequences to be used with a particular insert can be carried out by, for example, the library screening method of the invention as described herein. Briefly, in this method, libraries are constructed that have random sequences in a region desired for identification of effective linker sequences. Viruses generated from the library are tested for viability and immunogenicity of the inserted sequences, to identify effective linkers.

### Heterologous Peptides

The viral vectors of the invention can be used to deliver any peptide or protein of prophylactic or therapeutic value. For example, the vectors of the invention can be used in the induction of an immune response (prophylactic or therapeutic) to any protein-based antigen that is inserted into an HRV protein.

The vectors of the invention can each include a single epitope. Alternatively, multiple epitopes can be inserted into the vectors, either at a single site (e.g., as a polytope, in which the different epitopes can be separated by a flexible linker, such as a polyglycine stretch of amino acids), at different sites (e.g., the different Nim sites), or in any combination thereof. The different epitopes can be derived from a single species of pathogen, or can be derived from different species and/or different genuses. The vectors can include multiple peptides, for example, multiple copies of peptides as listed herein or combinations of peptides such as those listed herein. As an example, the vectors can include human and avian M2e peptides (and/or consensus sequences thereof).

Antigens that can be used in the invention can be derived from, for example, infectious agents such as viruses, bacteria, and parasites. A specific example of such an infectious agent is influenza viruses, including those that infect humans (e.g., A, B, and C strains), as well as avian influenza viruses. Examples of antigens from influenza viruses include those derived from M2, hemagglutinin (HA; e.g., any one of H1-H16, or subunits thereof) (or HA subunits HA 1 and HA2), neuraminidase (NA; e.g., any one of N1-N9), M1, nucleoprotein (NP), and B proteins.

Additional sequences that can be included in the vectors of the invention are influenza virus M2e sequences. Examples of such sequences are provided throughout this specification and in Sequence Appendix 1. Specific examples of such sequences include the following: MSLLTEVETPIRNEWGCRCNDSSD (SEQ ID NO:1); MSLLTEVETPTRNEWECRCSDSSD (SEQ ID NO:5); MSLLTEVETLTRNGWGCRCSDSSD (SEQ ID NO:6); EVETPTRN (SEQ ID NO:2); SLLTEVETPIRNEWGCRCNDSSD (SEQ ID NO:7); and SLLTEVETPIRNEWGCR (SEQ ID NO:8). Additional M2e sequences that can be used in invention include sequences from the extracellular domain of BM2 protein of influenza B (consensus MLEPFQ (SEQ ID NO:9)), and the M2e peptide from the H5N1 avian flu (MSLLTEVETLTRNGWGCRCSDSSD (SEQ ID NO:6)).

The peptides included in the vectors of the invention can include the complete sequences, noted above, or fragments including epitopes capable of inducing the desired immune response. Such fragments may include, e.g., 2-20, 3-18, 4-15, 5-12, or 6-10 amino acid fragments from within these peptides. Further, additional amino and/or carboxyl terminal amino acid sequences can be included in such peptides. Thus, the peptides can include, e.g., 1-10, 2-9, 3-8, 4-7, or 5-6 such amino acids, whether of naturally occurring, contiguous sequences, or artificial linker sequences (also see below). All such possible peptide fragments of the sequences noted above are included in the invention.

Other examples of peptides that are conserved in influenza can be used in the invention and include the NBe peptide conserved for influenza B (consensus sequence MNNATFNYTNVNPISHIRGS (SEQ ID NO:10)). Further examples of influenza peptides that can be used in the invention, as well as proteins from which such peptides can be derived (e.g., by fragmentation) are described in US 2002/0165176, US 2003/0175290, US 2004/0055024, US 2004/0116664, US 2004/0219170, US 2004/0223976, US 2005/0042229, US 2005/0003349, US 2005/0009008, US 2005/0186621, U.S. Pat. No. 4,752,473, U.S. Pat. No. 5,374,717, U.S. Pat. No. 6,169,175, U.S. Pat. No. 6,720,409, U.S. Pat. No. 6,750,325, U.S. Pat. No. 6,872,395, WO 93/15763, WO 94/06468, WO 94/17826, WO 96/10631, WO 99/07839, WO 99/58658, WO 02/14478, WO 2003/102165, WO 2004/053091, WO 2005/055957, and the enclosed Sequence Appendices 1 and 2 (and references cited therein), the contents of which are incorporated herein by reference. Further, conserved immunologic/protective T and B cell epitopes of influenza can be chosen from the www.immuneepitope.org database, in which many promising cross-protective epitopes have been recently identified (Bui et al., Proc. Natl. Acad. Sci. U.S.A 104:246-251, 2007 and supplemental tables). The invention can employ any peptide from the on-line IEDB resource can be used, e.g., influenza virus epitopes including conserved B and T cell epitopes described in Bui et al., supra.

Protective epitopes from other human/veterinary pathogens, such as parasites (e.g., malaria), other pathogenic viruses (e.g., human papilloma virus (HPV), herpes simplex viruses (HSV), human immunodeficiency viruses (HIV; e.g., gag), and hepatitis C viruses (HCV)), and bacteria (e.g., Mycobacterium tuberculosis, Clostridium difficile, and Helicobacter pylori) can also be included in the vectors of the invention. Various appropriate epitopes of these and other pathogens can be easily found in the literature. For example, cross-protective epitopes/peptides from papillomavirus L2 protein inducing broadly cross-neutralizing antibodies that protect from different HPV genotypes have been identified by Schiller and co-workers, such as amino acids 1-88, amino acids 1-200, or amino acids 17-36 of L2 protein of, e.g., HPV16 virus (WO 2006/083984 A1; QLYKTCKQAGTCPPDIIPKV (SEQ ID NO:11)). Examples of additional pathogens, as well as antigens and epitopes from these pathogens, which can be used in the invention are provided in WO 2004/053091, WO 03/102165, WO 02/14478, and US 2003/0185854, the contents of which are incorporated herein by reference.

Additional examples of pathogens from which antigens can be obtained are listed in Table 1, below, and specific examples of such antigens include those listed in Table 2. In addition, specific examples of epitopes that can be inserted into the vectors of the invention are provided in Table 3. As is noted in Table 3, epitopes that are used in the vectors of the invention can be B cell epitopes (i.e., neutralizing epitopes) or T cell epitopes (i.e., T helper and cytotoxic T cell-specific epitopes).

The vectors of the invention can be used to deliver antigens in addition to pathogen-derived antigens. For example, the vectors can be used to deliver tumor-associated antigens for use in immunotherapeutic methods against cancer. Numerous tumor-associated antigens are known in the art and can be administered according to the invention. Examples of cancers (and corresponding tumor associated antigens) are as follows: melanoma (NY-ESO-1 protein (specifically CTL epitope located at amino acid positions 157-165), CAMEL, MART 1, gp100, tyrosine-related proteins TRP1 and 2, and MUC1); adenocarcinoma (ErbB2 protein); colorectal cancer (17-1A,791Tgp72, and carcinoembryonic antigen); prostate cancer (PSA1 and PSA3). Heat shock protein (hsp110) can also be used as such an antigen.

In another example of the invention, exogenous proteins that encode an epitope(s) of an allergy-inducing antigen to which an immune response is desired can be used. In addition, the vectors of the invention can include ligands that are used to target the vectors to deliver peptides, such as antigens, to particular cells (e.g., cells that include receptors for the ligands) in subjects to whom the vectors administered.

The size of the peptide or protein that is inserted into the vectors of the invention can range in length from, for example, from 3-1000 amino acids in length, for example, from 5-500, 10-100, 20-55, 25-45, or 35-40 amino acids in length, as can be determined to be appropriate by those of skill in the art. Thus, peptides in the range of 10-25, 12-22, and 15-20 amino acids in length can be used in the invention. Further, the peptides noted herein can include additional sequences or can be reduced in length, also as can be determined to be appropriate by those skilled in the art. The peptides listed herein can be present in the vectors of the invention as shown herein, or can be modified by, e.g., substitution or deletion of one or more amino acids (e.g., 1, 2, 3, 4, 5, 6, 7, 8, 9, 10, or more amino acids). In addition, the peptides can be present in the vectors in the context of larger peptides. Optionally, peptides such as those described above and elsewhere herein include additional sequences on the amino and/or carboxyl terminal ends, as discussed above, whether such sequences are naturally associated with the peptide sequences (i.e., the sequences with which the peptides are contiguous in the influenza virus (or other source) genome) or not (e.g., synthetic linker sequences). The peptides can thus include, e.g., 1-25, 2-20, 3-15, 4-10, or 4-8 amino acid sequences on one or both ends. As a specific example, the peptide may include 1-3 linker sequences at amino and/or carboxyl terminal ends.

### Administration

When used in immunization methods, the vectors of the invention can be administered as a primary prophylactic agent in adults or children at risk of infection by a particular pathogen, such as influenza virus. The vectors can also be used as secondary agents for treating infected patients by stimulating an immune response against the pathogen from which the peptide antigen is derived. In the context of immunization against cancer, the vaccines can be administered against subjects at risk of developing cancer or to subjects that already have cancer.

For vaccine applications, optionally, adjuvants that are known to those skilled in the art can be used. Adjuvants are selected based on the route of administration. In the case of intranasal administration, chitin microparticles (CMP) can be used (Asahi-Ozaki et al., Microbes and Infection 8:2706-2714, 2006; Ozdemir et al., Clinical and Experimental Allergy 36:960-968, 2006; Strong et al., Clinical and Experimental Allergy 32:1794-1800, 2002). Other adjuvants suitable for use in administration via the mucosal route (e.g., intranasal or oral routes) include the heat-labile toxin of E. coli (LT) or mutant derivatives thereof. In the case of inactivated virus, parenteral adjuvants can be used including, for example, aluminum compounds (e.g., an aluminum hydroxide, aluminum phosphate, or aluminum hydroxyphosphate compound), liposomal formulations, synthetic adjuvants, such as (e.g., QS21), muramyl dipeptide, monophosphoryl lipid A, or polyphosphazine.

In addition, genes encoding cytokines that have adjuvant activities can be inserted into the vectors of the invention. Thus, genes encoding cytokines, such as GM-CSF, IL-2, IL-12, IL-13, or IL-5, can be inserted together with foreign antigen genes to produce a vaccine that results in enhanced immune responses, or to modulate immunity directed more specifically towards cellular, humoral, or mucosal responses. Alternatively, cytokines can be delivered, simultaneously or sequentially, separately from a recombinant vaccine virus by means that are well known (e.g., direct inoculation, naked DNA, in a viral vector, etc.).

The viruses of the invention can be used in combination with other vaccination approaches. For example, the viruses can be administered in combination with subunit vaccines including the same or different antigens. The combination methods of the invention can include co-administration of viruses of the invention with other forms of the antigen (e.g., subunit forms or delivery vehicles including hepatitis core protein (e.g., hepatitis B core particles containing M2e peptide on the surface produced in E. coli (HBc-M2e; Fiers et al., Virus Res. 103:173-176, 2004; WO 2005/055957; US 2003/0138769 A1; US 2004/0146524A1; US 2007/0036826 A1)), or inactivated whole or partial virus). Alternatively, the vectors of the present invention can be used in combination with other approaches (such as subunit or HBc approaches) in a prime-boost strategy, with either the vectors of the invention or the other approaches being used as the prime, followed by use of the other approach as the boost, or the reverse. Further, the invention includes prime-boost strategies employing the vectors of the present invention as both prime and boost agents. Thus, such methods can involve an initial administration of a vector according to the invention, with one or more (e.g., 1, 2, 3, or 4) follow-up administrations that may take place one or more weeks, months, or years after the initial administration.

The vectors of the invention can be administered to subjects, such as mammals (e.g., human subjects) using standard methods. In the case of intranasal administration, the vectors can be administered in the form of nose-drops or by inhalation of an aerosolized or nebulized formulation. The viruses can be in lyophilized form or dissolved in a physiologically compatible solution or buffer, such as saline or water. Standard methods of preparation and formulation can be used as described, for example, in Remington's Pharmaceutical Sciences (18^{th} edition), ed. A. Gennaro, 1990, Mack Publishing Company, Easton, Pa. Further, determination of an appropriate dosage amount and regimen can readily be determined by those of skill in the art.

The vectors of the invention can be administered to subjects, such as humans, as live or killed vaccines. The live vaccines can be administered intranasally using methods known to those of skill in the art (see, e.g., Grünberg et al., Am. J. Respir. Crit. Car. Med. 156:609-616, 1997). Appropriate dosage amounts and regimens can readily be determined by those of skill in the art. As an example, the dose range can be, e.g., 10³ to 10⁸ pfu per dose. The vaccine can advantageously be administered in a single dose, however, boosting can be carried out as well, if determined to be necessary by those skilled in the art. As to inactivated vaccines, the virus can be killed with, e.g., formalin or UV treatment, and administered intranasally at about 10⁸ pfu per dose, optionally with appropriate adjuvant (e.g., chitin or mutant LT; see above). In such approaches, it may be advantageous to administer more than one (e.g., 2-3) dose.

The invention is based, in part, on the following experimental examples.

### Experimental Examples

### I. Construction of HRV14-NimII-M2e Chimeras

We have constructed HRV14 NimII-M2e recombinant viruses. The viruses have been shown to express M2e on the virion surface, as demonstrated by the ability of anti-M2e Mab to neutralize the infectivity of the recombinant viruses.

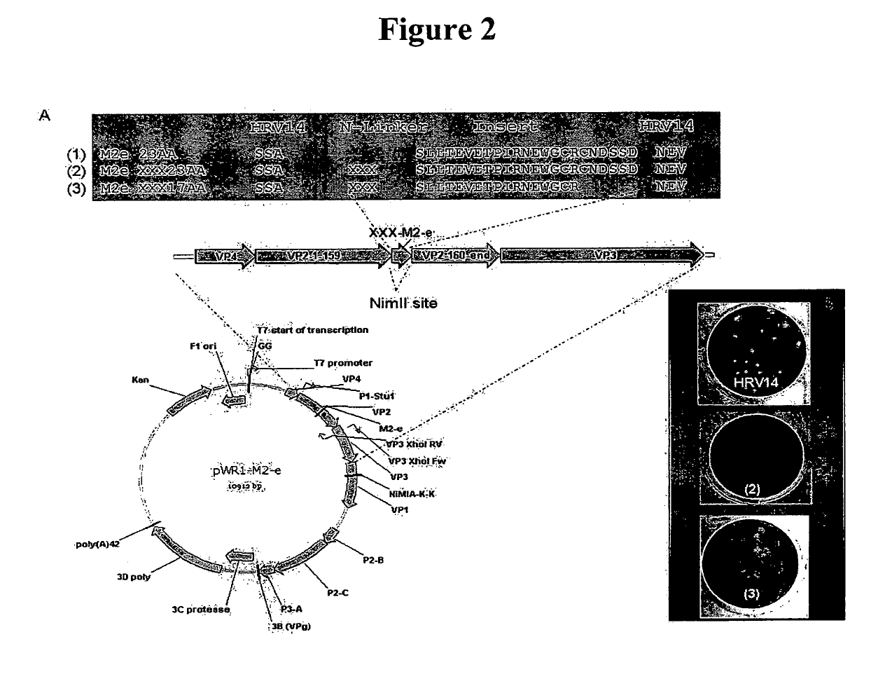

Three types of HRV14-M2e constructs were created (FIG. 2).

1. HRV14-NimII-23AA carrying the 23 AA of M2e inserted between AA159 and 160 of VP2 (NimII site);

2. HRV14-NimII-XXX23AA library. This set of constructs (plasmid library) was similar to the first construct except for the presence of a 3-AA randomized N-terminal linker fused to the peptide. This randomized linker was generated by the M2e sequence using a 5′ (direct) primer containing 9 randomized nucleotides coding for the linker amino acids; and

3. HRV14-NimII-XXX17AA library. This library was generated the same way as the first, but contained a shortened M2e peptide containing only the first 17 AA of M2e.

To facilitate the cloning process into the HRV14 infectious clone, we modified the pWR3.26 infectious clone by replacing its pUC plasmid backbone with that of the pEt vector (Novagen). Resulting plasmid pWR1 (FIG. 2) is more stably maintained in E. coli and easier to manipulate. Plaque morphology of virus libraries #2 and #3 differed from that of the HRV14 parent (FIG. 2B). The plaque size of the libraries appeared to be similar to wild type, but plaques were opaque. Construct #1 did not form plaques upon transfection.

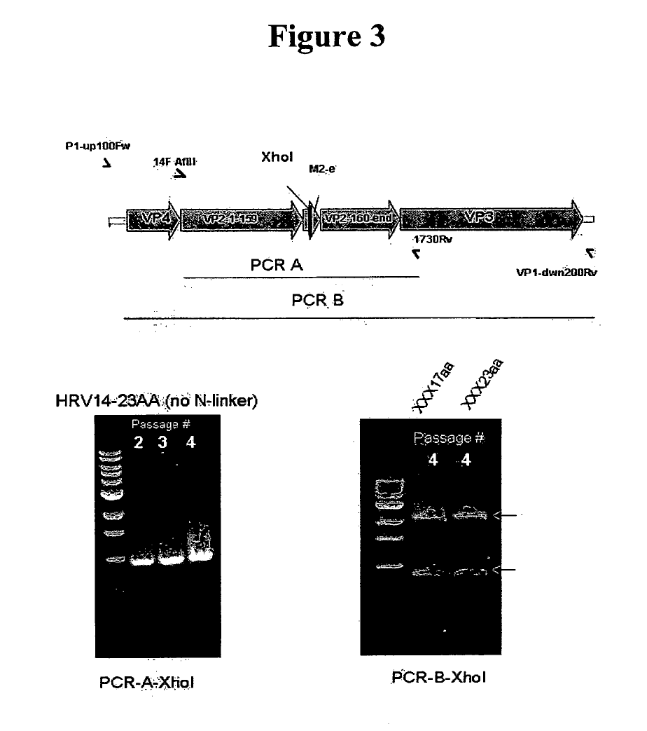

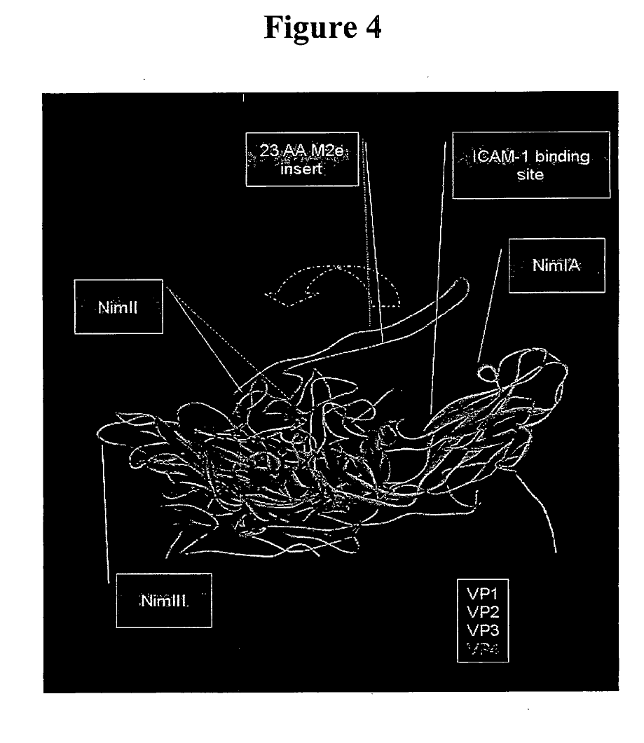

To monitor genetic stability of the constructed viruses, we incorporated an XhoI cleavage site in the middle of the M2e sequence by silent mutagenesis. An RT-PCR fragment obtained from virus containing mutated M2e gene is cleaved by XhoI, while the corresponding DNA product produced on wild type HRV14 remains undigested (FIG. 3). HRV14-NimII-23AA chimeric construct (#1) resulted in viable, but rather unstable virus. As shown in FIG. 3, the two XhoI digestion products of “PCR A” fragment are detectable only at passage 2, but not at following passages. Libraries (#2) and (#3), on the contrary, stably maintained the M2e insert: fragments “PCR B” obtained from virus libraries at the 4^{th} passage in H1 HeLa cells were completely digested by XhoI (FIG. 3). The instability of construct #1 could be due to steric interference of the inserted peptide with the receptor binding domain (FIG. 4), which may be alleviated when a degenerate linker is provided, as in constructs #2 and #3. The randomized N-terminal linker may have redirected the peptide away from the canyon containing the receptor binding domain allowing efficient virus binding to its receptor (FIG. 4).

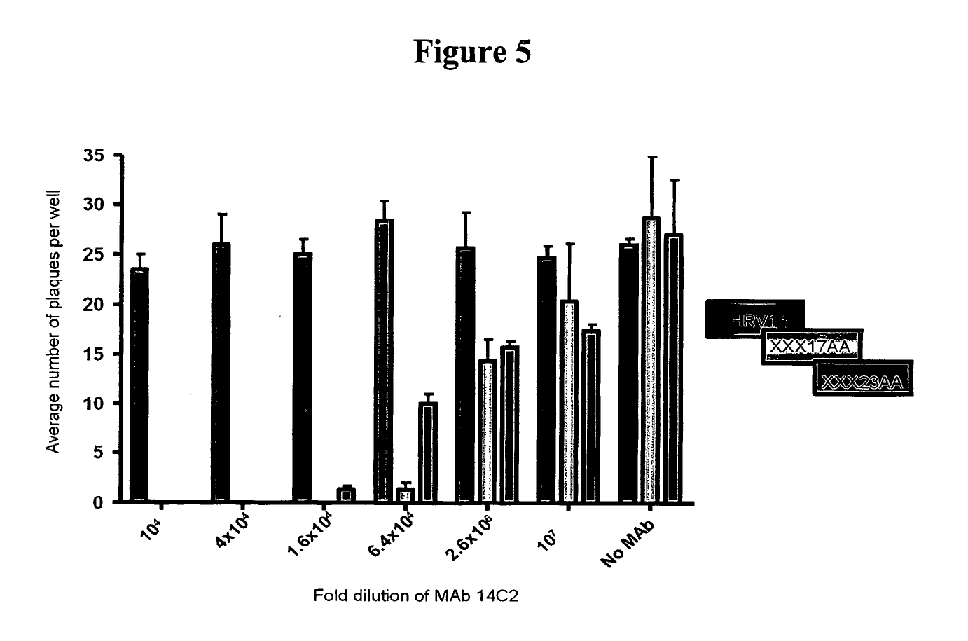

We carried out neutralization studies with the virus libraries with an anti-M2e Mab (14C2 MAb, Abcam, Inc. Cat# ab5416). Virus neutralization can be also used as a tool to demonstrate purity of libraries (i.e., the absence of wild type HRV14). The results of a plaque reduction neutralization test (PRNT) demonstrated extremely high specificity and neutralizing ability of Mab 14C2 against both libraries (FIG. 5).

Both libraries were shown to be extremely susceptible to neutralization by the anti-M2e Mab (FIG. 5), while control virus (pWR1) was not neutralized, even at the lowest dilution of 1:10 of the Mab. Fifty-percent neutralization for both libraries was observed at ˜1:2,000,000 dilution of antibody (stock concentration of 14C2 was 1 mg/ml). Such an efficient neutralization of the recombinant viruses showed that the M2e peptide presented in NimII of HRV14 is in an appropriate conformation, easily recognizable by antibody.

### II. Identification of Stable HRV14-NimII-M2e Recombinants

After 4 passages in H1 HeLa cells, six individual clones from each library were plaque purified and, after an additional 4 passages, characterized by sequencing of the carried insert. Each library gave rise to one dominant and stably replicating viral clone. All viruses isolated from HRV14-NimII-XXX23AA library had the same insert sequence, GHTSLLKEVETPIRNEWGSRSNDSSD (SEQ ID NO:12) with GHT as an N-terminal linker, whereas all of the viruses from the HRV14-NimII-XXX17AA library exhibited the same sequence, QPASLLTEVETPIRNEWGSR (SEQ ID NO:13), but with QPA as the N-terminal linker. All viable clones carrying the 23 AA insert had a substitution at position amino acid 7 from a tyrosine to lysine (position 4 in the M2e foreign insert). The clones carrying the 17 AA insert all contained wild type M2e sequence. These results indicate that genetically stable recombinant HRV-M2e viruses can be isolated. In further in vivo studies, the potential of HRV14-M2e(17AA) to provide protection against PR8 strain of Influenza A was evaluated using intraperitoneal route of administration.

### III. In Vivo Study with HRV14-NimII-M2e Recombinants

### A. In Vivo Experiment #1: Intraperitoneal Immunization

### 1. Experimental Design

9 week old female Balb/c mice (8 mice per group) were primed on day 0, then boosted on day 21 by intraperitoneal administration with either sucrose purified HRV14-M2e(17AA; see a note (4) to Table 4) virus at 5.0×10⁶ pfu of HRV14-M2e(17 AA), 1.3×10⁷ pfu of parental HRV14, or mock (PBS) as negative controls, mixed with 100 μg of adjuvant (aluminum hydroxide) in a 500 μL volume. As a gold standard, a current vaccine candidate ACAM-FluA (recombinant Hepatitis B core particles carrying 3 copies of M2e) was used. The latter was used alone or in combination with HRV14-M2e or HRV14 for prime/boost (Table 4). To demonstrate protection, all mice were subjected to challenge with 4 LD₅₀ of influenza A/PR/8/34 (H1N1) virus on day 35. Morbidity and mortality were monitored for 21 days. To test for serum antibody against the carried peptide, mice were bled prior to inoculation (baseline) and again on day 33. M2e-specific antibody titers in sera were determined by an established ELISA performed in microtiter plates coated with synthetic M2e peptide. Titers of M2e-specific total IgG, Ig2a, and Ig2b were determined.

### 2. Results

a. Immunogenicity
i. Total IgG in Immunized Animals

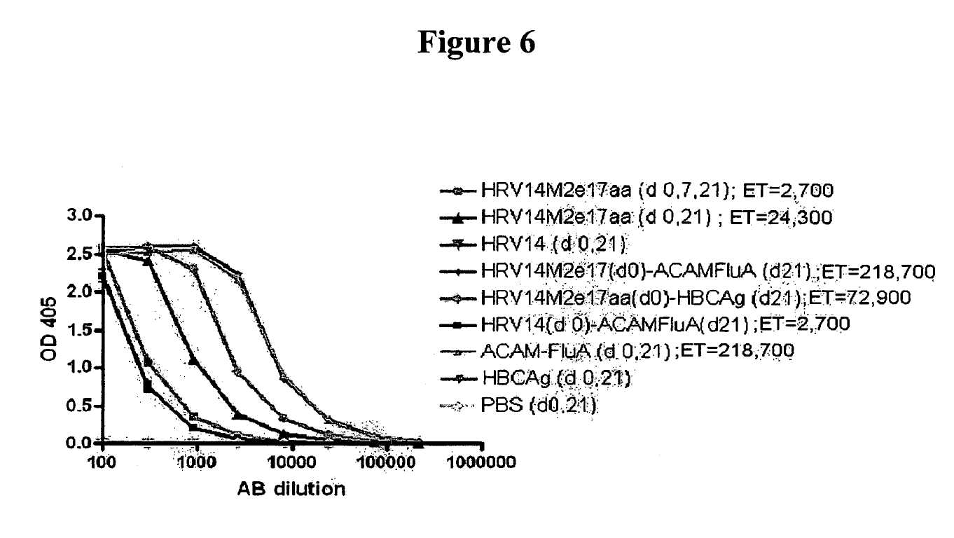

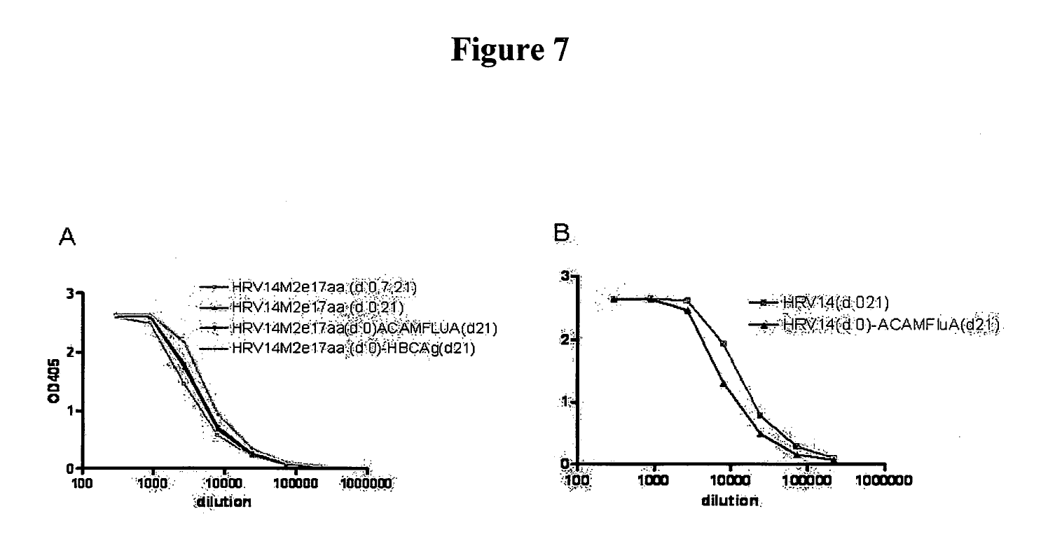

M2e-specific antibody titers were measured for each group using pooled serum samples (FIG. 6) as well as individual animal samples (FIG. 7). The results with pooled samples (FIG. 6) showed that prime with recombinant HRV14 carrying the 17 AA M2e and boost with ACAM-FluA elicited the same level of antibodies as two doses of Hepatitis B virus core-M2e recombinant virus-like particles (10 μg/dose) (end point titer (ET)=218,700). Boost with ACAM-FluA elicited about 100 times higher M2-e specific response when primed with HRV14-M2e(17AA) (group 4; ET=218,700) than with HRV14 vector (group 6; ET=2,700). Thus, the priming effect of HRV14-M2e is solely dependent on M2e insert and not on vector.

Based on the assumption made by Arnold et al., 2006 (Arnold, G. F. and Arnold, E. Chimeric Virus Vaccine. Ser. No. 11/176,182 [US 2006/0088549 A1], 1-57. Apr. 27, 2006. US. Jul. 7, 2005) an immunizing dose of 10⁹ pfu of HRV14 corresponds to approximately 10 μg of protein. We have roughly estimated that one immunizing dose of recombinant HRV-M2e virus represents 10 ng of protein. Taking into account differences in molecular mass and the multiplicity of subunits in the recombinant Hepatitis B core particles, we speculate that one immunizing dose of HBc-M2e contained approximately 10,000 times more M2e protein than that of HRV-M2e. Comparable antibody levels using HRV vectors perhaps supports a more immunogenic presentation system using a cheaper production methodology.

The level of M2e antibodies was inversely proportional to a number of doses of HRV14-M2e(17AA). Indeed three doses of HRV14-M2e(17AA) virus (group 1) elicited the lowest M2-e specific response (ET=2.700), whereas two dose regiment elicited 10 times higher (group 2; ET=24, 300) and one dose 3 times higher then two doses (group 5; ET=72,900). To verify whether this correlation is due to anti-vector immunity, we tested separately immune response of all groups to HRV14 vector (FIG. 7). All three types of administrations of HRV14-M2e(17AA) (1, 2, or 3 doses) showed comparable levels of HRV14-specific response (ET=72,900) (FIG. 7A). This argues against anti-vector immunity as a reason for decreased immune response to M2-e and suggests that of one-dose administration might be sufficient.

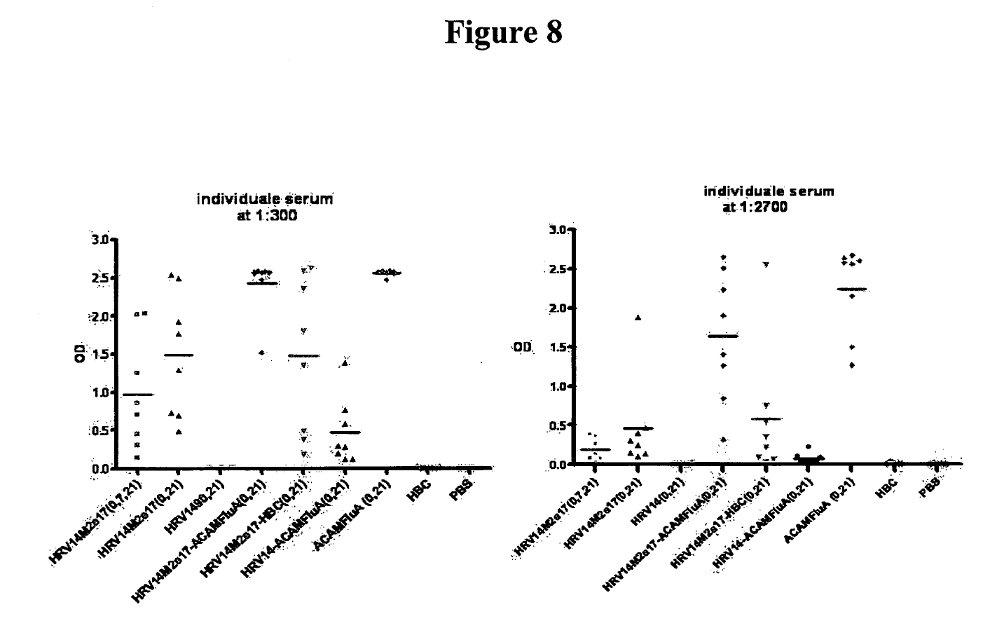

M2e-specific ELISA of individual serum samples (FIG. 8) detected the same intra-group differences as were shown with pooled samples: the average antibody levels in individual mice of groups 4 and 7 were significantly higher than for any other group studied as was shown at two serum dilutions (1:300 and 1:2,700)

ii. IgG2a, IgG2b, and IgG1 Subtypes of Antibodies in Immunized Animals

The dominant M2-specific Ab isotype in M2e vaccinated mice was shown to be IgG2b with some IgG2a (Jegerlehner et al., J. Immunol. 172.9:5598-5605, 2004). These two isotypes have been shown to be the most important mediators of antibody-dependent cytotoxicity (ADCC) in mice (Denkers et al., J. Immunol. 135:2183, 1985), which is believed is the major mechanism for M2e-dependent protection. In this study we have tested pooled group and individual sera samples for IgG1, IgG2a, and IgG2b isotype titers.

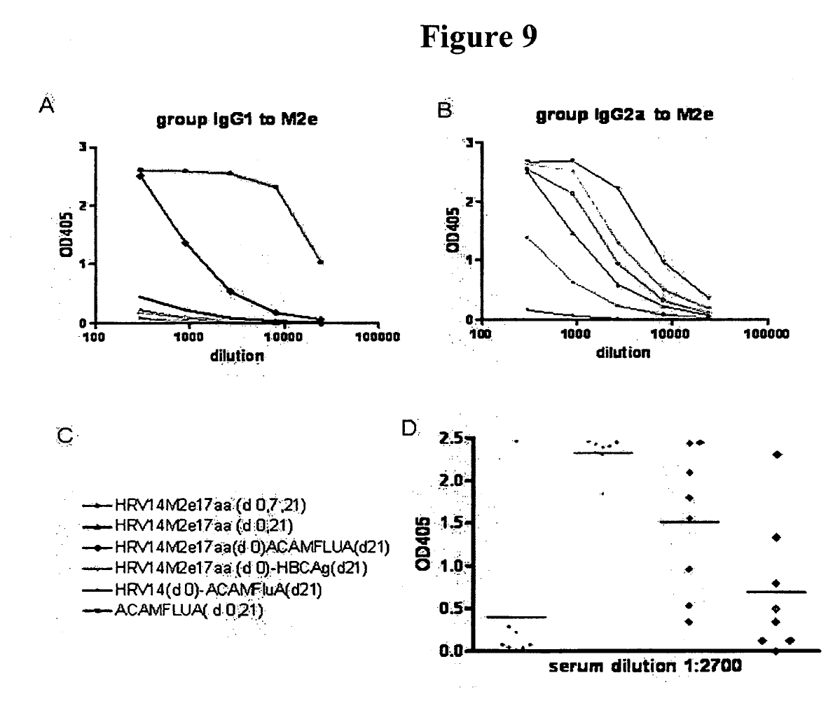

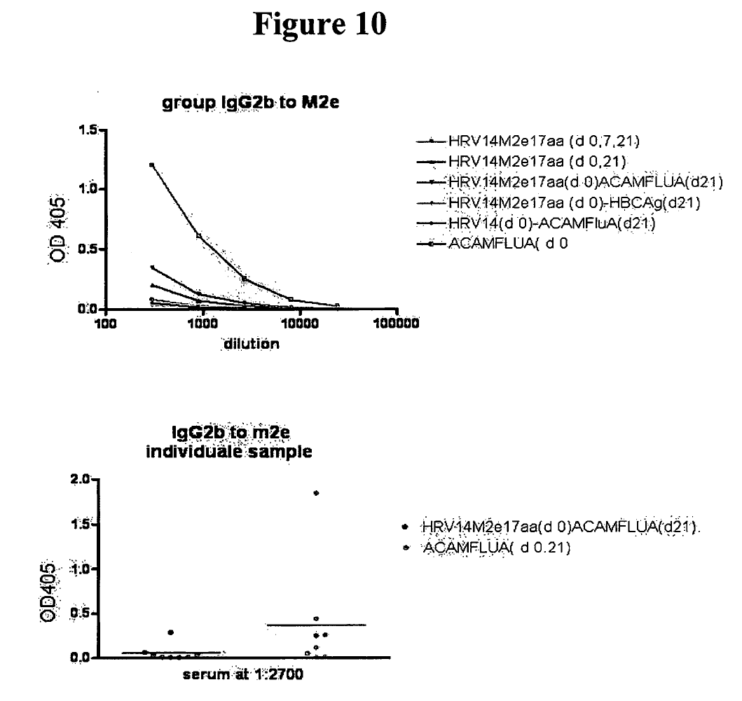

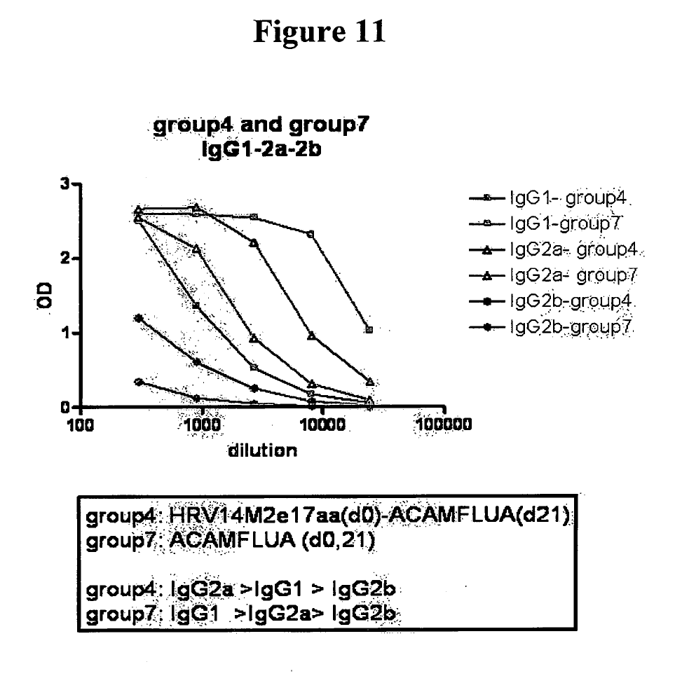

Groups 4 (prime with HRV14-M2e (17AA)/boost with ACAM-FluA) and 7 (prime/boost with ACAM-FluA) demonstrated the highest titers of IgG1 and IgG2a antibodies among other groups (FIG. 9). IgG1 titers were significantly higher in group 7 than in group 4 (FIGS. 9A and 9D), whereas IgG2a titers were higher in group 4 (FIGS. 9B and 9D), whereas IgG2b titers of group 7 animals were higher than in group 4 (FIG. 10). M2e-specific antibody of IgG2a isotype in mice immunized is shown in FIG. 11.

b. Morbidity and Mortality

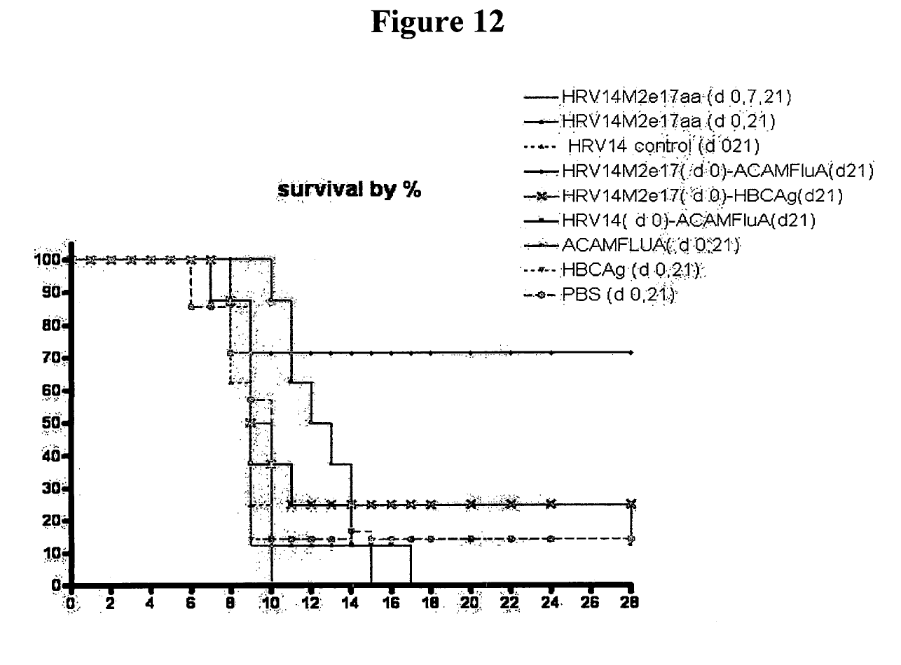

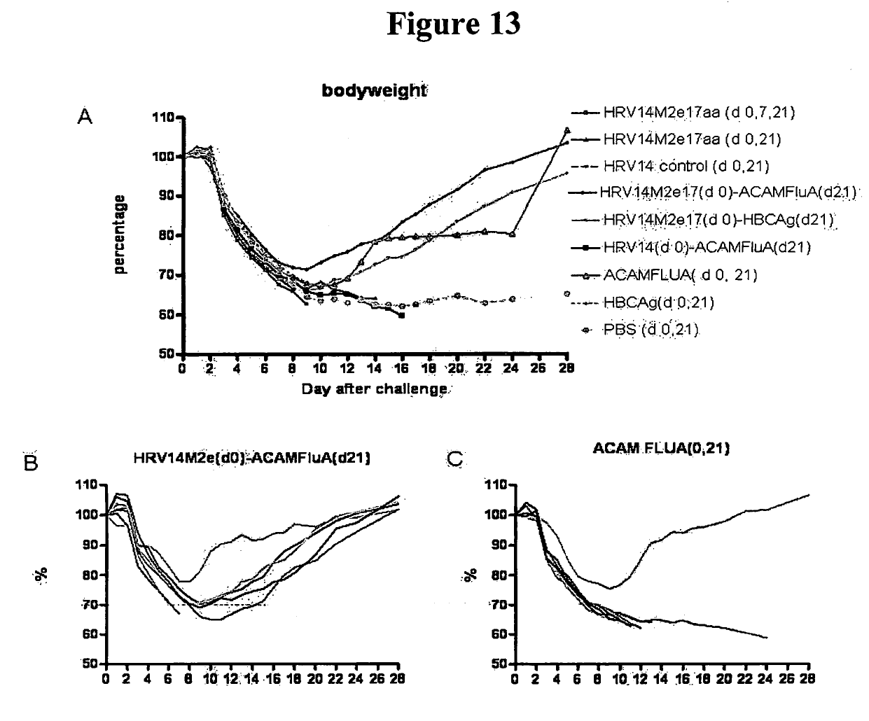

Mice were monitored for morbidity and mortality for 28 days after challenge with PR8 strain. As is shown in FIG. 12, group 4 demonstrated the highest survival rate (80%) in comparison to all other groups studied, whereas group 7 showed no significant difference with negative control (PBS). Group 4 was also a champion by morbidity: the body weight changes were significantly less dramatic than in all other groups (FIG. 13A, B).

Thus, HRV14-M2e (17 AA) virus is highly immunogenic and protective in mice. It compares responses to the traditional recombinant protein regimen and a combination of the two in a prime-boost regimen. The latter demonstrated a significantly different immune response than recombinant protein alone: two doses of recombinant HBc carrying M2e (Acam-FluA) elicited dominant IgG1 antibody subtype, whereas prime with HRV14-M2e(17AA) and boost with Acam-FluA generated IgG2a as a dominant isotype, which was shown to be important for ADCC. Moreover, the latter group demonstrated highest protection over all other groups.

It is important to note that because HRV does note replicate in mice, inoculation of HRV-M2e recombinants in this model is with a suitable parenteral adjuvant and mimics immunization with an inactivated vaccine. We propose to ultimately evaluate in humans, two options: live recombinant HRV14-M2e virus vaccine and/or inactivated vaccine (e.g., formalin-inactivated) co-administered with a licensed parenteral adjuvant such as aluminum hydroxide.

### B. In Vivo Experiment #2. Intranasal Immunization

### 1. Viruses Used for Immunization

In this in vivo study, the potential of HRV14-M2e (17AA) to provide protection against non-mortal challenge with PR8 strain of Influenza A was evaluated using intranasal route of administration. Note: The HRV14-M2e (17AA) sequence was described above.

2. Experimental Design 9 week old female Balb/c mice (8 mice per group) were primed on day 0, then boosted on days 21 by intranasal administration with either sucrose purified HRV14-M2e(17AA) or HRV14 (see a note (3) to Table 5) virus at 10⁸ pfu per dose (groups 3-6), mixed with 5 μg of Heat-Labile Toxin of E. coli (LT) adjuvant in a 50 μL volume. As a gold standard a vaccine comprising recombinant Hepatitis B core particles carrying 3 copies of M2e (AcamFluA) was used. The latter was used alone or in combination with HRV14-M2e or HRV14 for prime/boost (Table 5). To demonstrate protection, all mice were subjected to challenge with 4 LD₅₀ of influenza A/PR/8/34 (H1N1) virus on day 35. Morbidity and mortality were monitored for 21 days. To test for serum antibody against the carried peptide, mice were bled prior to inoculation (baseline) and again on day 33. M2e-specific antibody titers in sera were determined by an established ELISA performed in microtiter plates coated with synthetic M2e. Titers of M2-e specific total IgG, Ig2a, and Ig2b were determined.

### 3. Results

a. Immunogenicity

i. M2e-Specific Antibody Titers

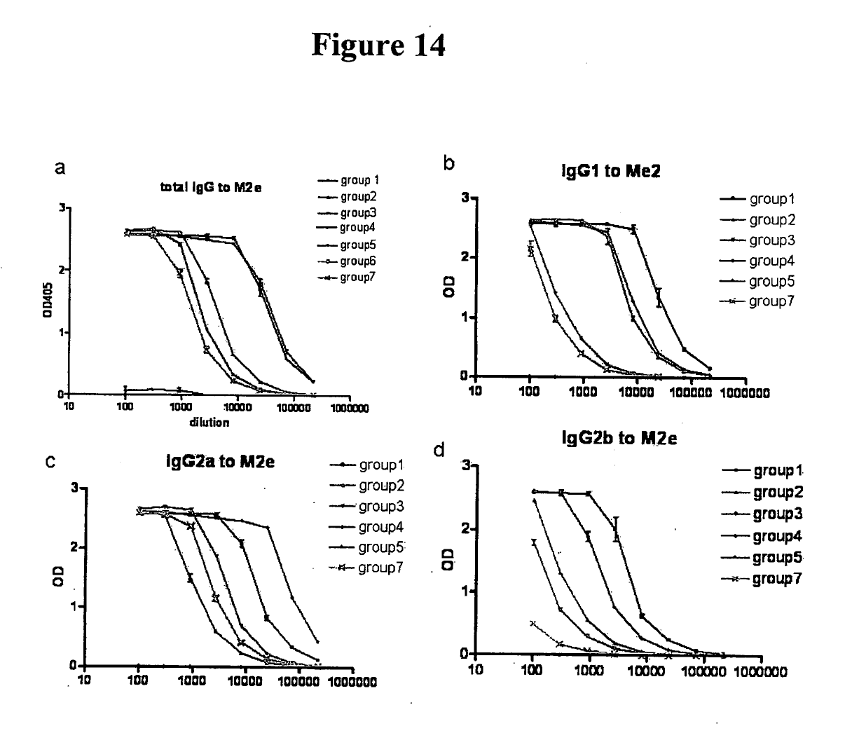

Antibody titers were measured for each group using pooled serum samples (FIG. 14). One dose of recombinant HRV14 carrying the 17 AA M2e and a boost with ACAM-FluA elicited comparable levels of total IgG as two doses of Hepatitis B virus core-M2e recombinant virus-like particles (10 ug/dose) (end point titer (ET)>218,700; FIG. 14A). Later results are consistent with data obtained with IP route of immunization. One dose of HRV14-M2e elicited comparable level of total M2e-specific total IgG as one dose of AcamFluA (ET=24,300). A two-fold decrease in HRV14-M2e virus load has had not much of an effect on total IgG level (group 7; ET−=24,300).

As in the case of IP administration, priming with HRV14-M2e and boosting with AcamFluA generated the highest level of IgG2a (FIG. 14C; ET>218,700). One dose of HRV14-M2e elicited slightly higher level of IgG2a than one dose of AcamFluA (ET=72,900 vs ET=24,300). The highest titers IgG2b (FIG. 14B) and IgG1 (FIG. 14D) were demonstrated for two doses of AcamFluA.

b. Morbidity

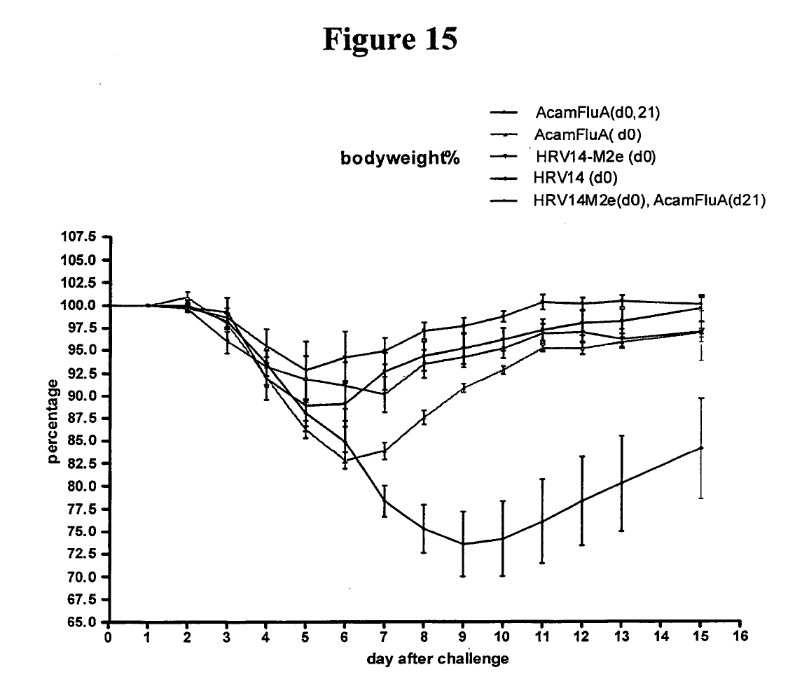

Mice were monitored for morbidity for 17 days after none-mortal challenge with PR8 strain (FIG. 15). One dose of HRV14-M2e provided the comparable protection from disease as two doses of AcamFluA or prime with HRV14-M2e and boost with AcamFluA. Mice in group 2 (one dose of AcamFluA) showed significant signs of disease. The control group (group 4) demonstrated severe body weight loss during first 9 days after challenge.

### IV. New Dominant Neutralizing Immunogen (NimIV) in HRV14 Virus, a Newly Discovered Insertion Site of Foreign Epitopes

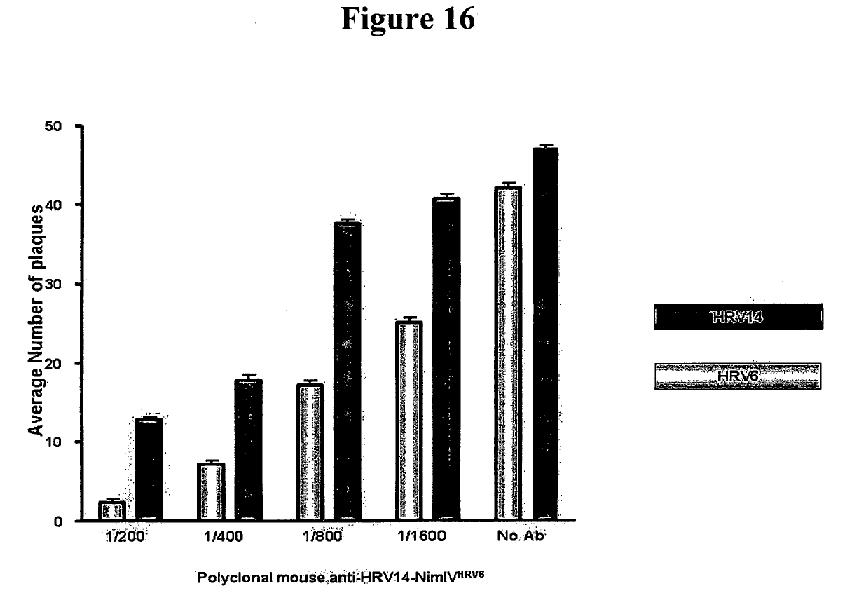

We have identified a new HRV neutralizing immunogen: Neutralizing Immunogen IV (NimIV). It can be used for the development of epitope-insertion recombinant vaccines. NimIV is highly immunogenic, inducing high virus neutralizing titers in mice. NimIV of HRVs involves a C-terminal region of the structural protein VP 1. This epitope can be exchanged between different HRV serotypes. If NimIV of one HRV is introduced into another serotype virus, it confers unto the resulting chimeric recombinant the neutralization characteristics of the donor serotype. Synthetic NimIV peptides were shown to be efficiently recognized by corresponding serotype-specific antibodies in ELISA and Western blot experiments. Specifically, an HRV14-NimIV^{HRV6} chimera was produced by replacing the NimIV^{HRV14} in HRV14 with NimIV from HRV6 virus. This virus was efficiently neutralized with anti-HRV6 polyclonal antibodies and also elicited anti-HRV6 neutralizing response in mice. The 50% neutralizing titer of sera from mice immunized with HRV14-NimIV^{HRV6} was ˜1:800 against HRV6 virus, and only 1:400 against HRV14 (FIG. 16). For comparison, 50% neutralization titer of mouse anti-HRV14 sera against homologous virus is 1:1400, showing that the HRV6-specific NimIV significantly reduced the effectiveness of virus neutralization by antibodies against the remaining HRV14 Nims (I, II, and III).

### V. Influenza Mouse Challenge Model

The protective efficacy of vaccine candidates can be tested in a mouse influenza challenge model using appropriate virus strains. The prototype influenza challenge strain used in our studies is mouse-adapted strain A/PR/8/34 (H1N1). The virus was obtained from the American Type Culture Collection (catalog number VR-1469, lot number 2013488) and adapted to in vivo growth by serial passage in Balb/c mice. For mouse passage, virus was inoculated intranasally and lung tissue homogenates were prepared 3 days later. The homogenate was blind-passaged in additional mice through passage 5. An additional passage was used to prepare aliquots of lung homogenate that serve as the challenge stock.

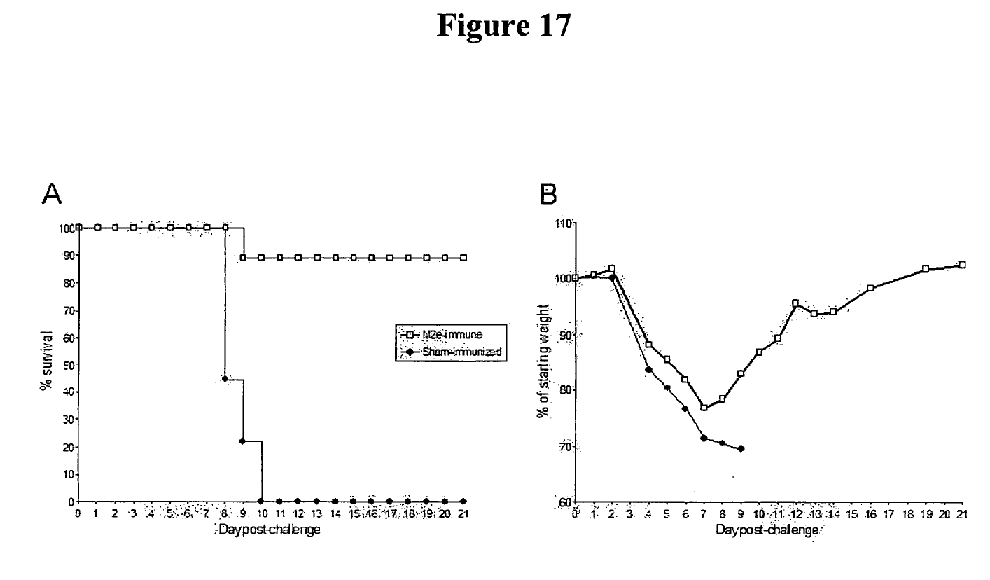

For challenge of mice, virus is delivered intranasally in a volume of 50 μl, The mice are anesthetized during inoculation to inhibit the gag reflex and allow passage of the virus into the lungs. Mice infected with a lethal dose of virus lose weight rapidly and most die 7-9 days after inoculation. The median lethal dose (LD₅₀) of mouse-adapted A/PR/8/34 virus was determined to be 7.5 plaque-forming units (pfu) in adult Balb/c mice. Results for a typical protection experiment are shown in FIG. 17. Groups of 10 mice were either sham-immunized with aluminum hydroxide adjuvant or immunized with 10 μg of influenza M2e peptide immunogen mixed with aluminum hydroxide. The immunogen consisted of hepatitis B core protein virus-like particles expressing M2e peptide. The mice were immunized twice at 3 week intervals and challenged intranasally 4 weeks later with 4 LD₅₀ of mouse-adapted A/PR/8/34 virus. All mice in the sham-immunized group died by the 10^{th} day after challenge, while only 1 mouse died in the immunized group. Loss in weight occurred after challenge in both groups, but was greater in the sham-immunized group.

Other influenza virus strains will be similarly adapted to growth in mouse lungs. In some cases strains may be used without in vivo adaptation or may not become sufficiently pathogenic even after serial lung passage. In this case, rather than measuring morbidity and mortality, we will measure virus replication in lung and nasal turbinate tissues. Tissues are harvested 3 days after challenge, disrupted by sonication in 1 ml of tissue culture medium and titrated for virus concentration by plaque or TCID₅₀ assay.

TABLE 1
List of examples of pathogens from which
epitopes/antigens/peptides can be derived

```
VIRUSES:
Flaviviridae
      Yellow Fever virus
      Japanese Encephalitis virus
      Dengue virus, types 1, 2, 3 & 4
      West Nile Virus
      Tick Borne Encephalitis virus
      Hepatitis C virus (e.g., genotypes 1a, 1b, 2a, 2b, 2c, 3a, 4a, 4b,
      4c, and 4d)
Papoviridae:
      Papillomavirus
Retroviridae
      Human Immunodeficiency virus, type I
      Human Immunodeficiency virus, type II
      Simian Immunodeficiency virus
      Human T lymphotropic virus, types I & II
Hepnaviridae
      Hepatitis B virus
Picornaviridae
      Hepatitis A virus
      Rhinovirus
      Poliovirus
Herpesviridae:
      Herpes simplex virus, type I
      Herpes simplex virus, type II
      Cytomegalovirus
      Epstein Barr virus
      Varicella-Zoster virus
Togaviridae
      Alphavirus
      Rubella virus
Paramyxoviridae:
      Respiratory syncytial virus
      Parainfluenza virus
      Measles virus
      Mumps virus
Orthomyxoviridae
      Influenza virus
Filoviridae
      Marburg virus
      Ebola virus
Rotoviridae:
      Rotavirus
Coronaviridae
      Coronavirus
Adenoviridae
      Adenovirus
Rhabdoviridae
      Rabiesvirus
BACTERIA:
Enterotoxigenic E. coli
Enteropathogenic E. coli
Campylobacter jejuni
Helicobacter pylori
Salmonella typhi
Vibrio cholerae
Clostridium difficile
Clostridium tetani
Streptococccus pyogenes
Bordetella pertussis
Neisseria meningitides
Neisseria gonorrhoea
Legionella neumophilus
Clamydial spp.
Haemophilus spp.
Shigella spp.
PARASITES:
Plasmodium spp.
Schistosoma spp.
Trypanosoma spp.
Toxoplasma spp.
Cryptosporidia spp.
Pneumocystis spp.
Leishmania spp.
```

TABLE 2
Examples of select antigens from listed viruses

| VIRUS | ANTIGEN |
|---|---|
| Flaviviridae | |
| Yellow Fever virus | Nucleocapsid, M & E glycoproteins |
| Japanese Encephalitis virus | ″ |
| Dengue virus, types 1, 2, 3 & 4 | ″ |
| West Nile Virus | ″ |
| Tick Borne Encephalitis virus | ″ |
| Hepatitis C virus | Nucleocapsid, E1 & E2 glycoproteins |
| Papoviridae: | |
| Papillomavirus | L1 & L2 capsid protein, E6 & E7 |
| | transforming protein (oncopgenes) |
| Retroviridae | |
| Human Immunodeficiency virus, | gag, pol, vif, tat, vpu, env, nef |
| type I | |
| Human Immunodeficiency virus, | ″ |
| type II | |
| Simian Immunodeficiency virus | ″ |
| Human T lymphotropic virus, | gag, pol, env |
| types I & II | |

TABLE 3
Examples of B and T cell epitopes from listed viruses/antigens

| | | | | |
|---|---|---|---|---|
| VIRUS | ANTIGEN | EPITOPE | LOCATION | SEQUENCE (5′-3′) |

Flaviviridae

| | | | | |
|---|---|---|---|---|
| Hepatitis C | Nucleocapsid | CTL | 2-9 | STNPKPQR |
| | | | | (SEQ ID NO: 14) |
| | | | 35-44 | YLLPRRGPRL |
| | | | | (SEQ ID NO: 15) |
| | | | 41-49 | GPRLGVRAT |
| | | | | (SEQ ID NO: 16) |
| | | | 81-100 | YPWPLYGNEGCGWAGWLLSP |
| | | | | (SEQ ID NO: 17) |
| | | | 129-144 | GFADLMGYIPLVGAPL |
| | | | | (SEQ ID NO: 18) |
| | | | 132-140 | DLMGYIPLV |
| | | | | (SEQ ID NO: 19) |
| | | | 178-187 | LLALLSCLTV |
| | | | | (SEQ ID NO: 20) |
| | E1 glycoprotein | CTL | 231-250 | REGNASRCWVAVTPTVATRD |
| | | | | (SEQ ID NO: 21) |
| | E2 glycoprotein | CTL | 686-694 | STGLIHLHQ |
| | | | | (SEQ ID NO: 22) |
| | | | 725-734 | LLADARVCSC |
| | | | | (SEQ ID NO: 23) |
| | | | 489-496 | CWHYPPRPCGI |
| | | | | (SEQ ID NO: 24) |
| | | | 569-578 | CVIGGVGNNT |
| | | | | (SEQ ID NO: 25) |
| | | | 460-469 | RRLTDFAQGW |
| | | | | (SEQ ID NO: 26) |
| | | | 621-628 | TINYTIFK |
| | | | | (SEQ ID NO: 27) |
| | | B cell | 384-410 | ETHVTGGNAGRTTAGLVGLL |
| | | | | TPGAKQN |
| | | | | (SEQ ID NO: 28) |
| | | | 411-437 | IQLINGSWHINSTALNCNES |
| | | | | LNTGW |
| | | | | (SEQ ID NO: 29) |
| | | | 441-460 | LFYQHKFNSSGCPERLASCR |
| | | | | (SEQ ID NO: 30) |
| | | | 511-546 | PSPVVVGTTDRSGAPTYSWGANDTDV |
| | | | | FVLNNTRPPL |
| | | | | (SEQ ID NO: 31) |
| | | T helper | 411-416 | IQLINT |
| | | | | (SEQ ID NO: 32) |
| Papoviridae | | | | |
| HPV 16 | E7 | T helper | 48-54 | DRAHYNI |
| | | | | (SEQ ID NO: 33) |
| | | CTL | 49-57 | RAHYNIVTF |
| | | | | (SEQ ID NO: 34) |
| | | B cell | 10-14 | EYMLD |
| | | | | (SEQ ID NO: 35) |
| | | | 38-41 | IDGP |
| | | | | (SEQ ID NO: 36) |
| | | | 44-48 | QAEPD |
| | | | | (SEQ ID NO: 37) |
| HPV 18 | E7 | T helper | 44-55 | VNHQHLPARRA |
| | | | | (SEQ ID NO: 38) |
| | | | 81-90 | DDLRAFQQLF |
| | | | | (SEQ ID NO: 39) |

TABLE 4
Immunization groups (Intraperitoneal Study)

| | Number | | | | |
|---|---|---|---|---|---|
| group | of | | | | Dosing |
| number | animals | Prime | Boost | Adj | (days) |
| 1 | 8 | HRV14- | HRV14- | Alum | 0, 7, 21 |
| | | M2e(17AA) | M2e(17AA) | | |
| 2 | 8 | HRV14- | HRV14- | Alum | 0, 21 |
| | | M2e(17AA) | M2e(17AA) | | |
| 3 | 8 | HRV14 | HRV14 | Alum | 0, 21 |
| 4 | 8 | HRV14- | ACAM-FluA | Alum | 0, 21 |
| | | M2e(17AA) | | | |
| 5 | 8 | HRV14- | HBcAg | Alum | 0, 21 |
| | | M2e(17AA) | | | |
| 6 | 8 | HRV14 | ACAM-FluA | Alum | 0, 21 |
| 7 | 8 | ACAM-FluA | ACAM-FluA | Alum | 0, 21 |
| 8 | 8 | HBcAg | HBcAg | Alum | 0, 21 |
| 9 | 8 | PBS | PBS | Alum | 0, 21 |

Notes for Table 4:
(1) ACAM-FluA - is a current universal Influenza A vaccine candidate based on Hepatitis B core antigen (HBc) carrying three copies of 23 AA M2-e peptide; used as a golden standard; the dose = 10 μg per mouse
(2) HBcAg is a “naked” HBc antigen; used as carrier control for ACAM-FluA; the dose = 10 μg per mouse
(3) HRV14 is “wild type” HRV14 produced from pWR3.26 infectious clone (ATCC); used as a carrier control for HRV14-M2e(17AA)
(4) HRV14M2e(17AA) is HRV14 virus carrying QPASLLTEVETPIRNEWGSR (SEQ ID NO: 13) sequence between 159 AA and 160AA of VP2 (NimII site). First three aminoacids (QPA) of this insert represent a unique linker selected from HRV14M2eXXX(17AA) library as described earlier
(5) ADJ = adjuvant (alum was used in all immunizations)
(6) All groups were immunized by intraperitoneal administration

TABLE 5
Immunization groups (Intranasal Study)

| | Number | | | | |
|---|---|---|---|---|---|
| group | of | | | | Dosing |
| number | animals | Prime | Boost | Adj | (days) |
| 1 | 8 | AcamFluA | AcamFluA | LT | 0, 21 |
| 2 | 8 | AcamFluA | | LT | 0 |
| 3 | 8 | HRV14- | | LT | 0 |
| | | M2e(17AA) | | | |
| 4 | 8 | HRV14 | | LT | 0 |
| 5 | 8 | HRV14- | AcamFluA | LT | 0, 21 |
| | | M2e(17AA) | | | |
| 6 | 8 | HRV14 | ACAM-FluA | LT | 0, 21 |

Notes for Table 5:
(1) ACAM-FluA - is an influenza A vaccine based on Hepatitis B core antigen (HBc) carrying three copies of 23 AA M2-e peptide; used as a gold standard; the dose = 10 μg per mouse
(2) HRV14 is “wild type” HRV14 produced from pWR3.26 infectious clone (ATCC); used as a carrier control for HRV14-M2e(17AA)
(3) HRV14M2e(17AA) is HRV14 virus carrying a QPASLLTEVETPIRNEWGSR (SEQ ID NO: 13) sequence between AA159 and AA160 of VP2 (NimII site). The first three amino acids (QPA) of this insert represent a unique linker selected from an HRV14M2eXXX(17AA) library as described above
(5) ADJ = adjuvant (LT = Heat-Labile Toxin of E. coli)
(6) All groups were immunized by Intranasal administration
(7) Groups 3, 4, 5, and 6 were immunized with correspondent viruses at 10⁸ pfu per dose

### Other Embodiments

All publications and patents cited in this specification are herein incorporated by reference as if each individual publication or patent were specifically and individually indicated to be incorporated by reference. Use of singular forms herein, such as “a” and “the,” does not exclude indication of the corresponding plural form, unless the context indicates to the contrary. Although the invention has been described in some detail by way of illustration and example for purposes of clarity of understanding, it will be readily apparent to those of ordinary skill in the art in light of the teachings of the invention that certain changes and modifications may be made thereto without departing from the spirit or scope of the appended claims.

Other embodiments are within the following claims.

1. A rhinovirus vector comprising an influenza virus antigen.

2. The rhinovirus vector of claim 1, wherein the rhinovirus vector is not pathogenic in humans.

3. The rhinovirus vector of claim 2, wherein the rhinovirus vector is Human Rhinovirus 14 (HRV14).

4. The rhinovirus vector of claim 1, wherein the influenza virus antigen comprises an M2e peptide.

5. The rhinovirus vector of claim 1, wherein the influenza antigen is inserted at the site of a neutralizing immunogen selected from the group consisting of Neutralizing Immunogen I (NimI), Neutralizing Immunogen II (NimII), Neutralizing Immunogen III (NimIII), and Neutralizing Immunogen IV (NimIV), or a combination thereof.

6. The rhinovirus vector of claim 5, wherein the influenza virus antigen is inserted at the site of Neutralizing Immunogen II (NimII).

7. The rhinovirus vector of claim 6, wherein the influenza antigen is inserted between amino acids 158 and 160 of NimII.

8. The rhinovirus vector of claim 1, wherein the influenza virus antigen is flanked by linker sequences on one or both ends.

9. The rhinovirus vector of claim 1, wherein the rhinovirus vector is live.

10. The rhinovirus vector of claim 1, wherein the rhinovirus vector is inactivated.

11. A pharmaceutical composition comprising the rhinovirus vector of claim 1 and a pharmaceutically acceptable carrier or diluent.

12. The pharmaceutical composition of claim 11, further comprising an adjuvant.

13. The pharmaceutical composition of claim 11, further comprising one or more additional active ingredients.

14. The pharmaceutical composition of claim 13, further comprising a Hepatitis B core protein fused with M2e sequences.

15. A method of inducing an immune response to an influenza virus in a subject, the method comprising administering to the subject the pharmaceutical composition of claim 11.

16. The method of claim 15, wherein the subject does not have but is at risk of developing influenza virus infection.

17. The method of claim 15, wherein the subject has influenza virus infection.

18. The method of claim 15, wherein the composition is administered to the subject intranasally.

19. The method of claim 15, wherein the subject is a human.

20. A method of making a pharmaceutical composition, comprising admixing the rhinovirus vector of claim 1 and a pharmaceutically acceptable carrier or diluent.

21. A nucleic acid molecule encoding or corresponding to the genome of the rhinovirus vector of claim 1.

22. A NimII peptide comprising an inserted influenza antigen.

23. A method of generating a rhinovirus vector comprising an influenza virus antigen, the method comprising the steps of:
(i) generating a library of recombinant rhinovirus vectors based on an infectious cDNA clone that comprises inserted influenza virus antigen sequences, and
(ii) selecting from the library recombinant viruses that (a) maintain inserted sequences upon passage, and (b) are neutralized with antibodies against the inserted sequence.

24. The method of claim 23, wherein the rhinovirus vector is human rhinovirus 14 (HRV14).

25. The method of claim 23, wherein the inserted influenza antigen sequence is inserted at a position selected from the group consisting of NimI, NimII, NimIII, and NimIV.

26. The method of claim 23, wherein the inserted influenza virus antigen sequence is an M2e sequence.

27. The method of claim 23, wherein the inserted influenza antigen sequence is flanked on one or both ends with random linker sequences.

28. A method of cultivating a rhinovirus vector comprising an influenza virus antigen, the method comprising passaging the vector in HeLa or MRC-5 cells.

29. A rhinovirus vector comprising a pathogen, cancer, or allergen-based antigen, as described herein.

30. A pharmaceutical composition comprising a rhinovirus vector of claim 29.

31. A method of inducing an immune response to an antigen from a pathogen, cancer, or allergen-based antigen, as described herein, the method comprising administration of a composition of claim 30.
